(19) United States
(12) Patent Application Publication
(10) Pub. No.: US 2024/0012040 A1
(43) Pub. Date: Jan. 11, 2024

(54) CORRELATION-BASED ENTROPY EXTRACTION SOLUTION FOR MIMO SYSTEMS

(71) Applicant: EV-TECHNOLOGIES, COLOMBELLES (FR)

(72) Inventor: Sidina WANE, Caen (FR)

(21) Appl. No.: 18/253,474

(22) PCT Filed: Nov. 25, 2021
(86) PCT No.: PCT/EP2021/083034

§ 371 Date: May 18, 2023

(30) Foreign Application Priority Data
Nov. 25, 2020 (EP) 20306441.5
Nov. 25, 2020 (EP) 20306444.9
Nov. 15, 2021 (WO) PCT/EP2021/081730

(51) Int. Cl. G01R 29/10; G01R 29/08; G01J 5/10

(52) U.S. Cl. CPC G01R 29/10; G01R 29/0871; G01J 5/10; A61B 5/01

(57) ABSTRACT

The present disclosure relates to an electromagnetic-thermal sensing system comprising: a conversion device (105) configured to receive one or more electromagnetic signals emitted by a DUT (102), the conversion device (105) comprising a thermal indicator layer (110) of quantum spin cross-over (SCO) material configured to change temperature as a function of an electrical and/or magnetic field present at the thermal indicator layer (110); and an imaging device (104) configured to capture one or more images of the conversion device (105).

The present patent application claims priority from the European patent applications filed on 25 Nov. 2020 and assigned application nos. EP20306444 and EP20306441, and from the International patent application filed on 15 Nov. 2021 and assigned application no. PCT/EP2021/081730, the contents of these three applications being hereby incorporated by reference.

### TECHNICAL FIELD

The present disclosure relates generally to imaging solutions for imaging and/or measuring microwave and millimeter-wave fields, for example in the context of testing and characterization of electronic devices, including radiating systems.

### BACKGROUND ART

The automatized testing and validation of 5G (Fifth Generation) and IoT (Internet of Things) communication devices requires appropriate instruments capable, for example, of evaluating power integrity (PI), signal integrity (SI), and conformity with EMC (Electro-Magnetic Capability) and EMI (Electro-Magnetic Interference) specifications. Indeed, PI, SI, EMC and EMI performance is a critical issue for new generation communications systems that are required to have very high data transmission rates, low energy consummation, and a strong immunity to undesirable disturbances.

The use of electromagnetic infrared techniques for visualizing and measuring microwave fields has been proposed, for example in the publication by T. Hasegawa entitled “A new method of observing electromagnetic fields at high frequencies by use of test paper”, Bull. Yamagata Univ. IV, Japan, 1995. The available techniques consist in inserting sensitive films with electric and/or magnetic properties, which induce currents resulting in heating, which can be recorded by the infrared cameras. However, a drawback of such available techniques is that they are based on materials that demand high input powers of up to several tens of dBm, and/or that lead to low heating effects that are very difficult to use for OTA (Over The Air) testing of devices and systems.

It has also been proposed to use high-sensitivity Spintronics sensors for near-field magnetic-field sensing of electronic circuits and radiating systems. Spintronic devices exploit the spin of electrons to generate and control charge currents, and to inter-convert electrical and magnetic signals. Spintronics sensors have advantages over other forms of sensors, such as coils, fluxgates and low-field sensing techniques, such as SQUIDs, thanks to their relatively small size and low power requirements.

However, there is a need to further improve existing solutions in terms of power-consumption, performance, complexity and cost.

### SUMMARY OF INVENTION

It is an aim of embodiments of the present disclosure to address one or more needs in the prior art.

According to one aspect, there is provided an electromagnetic-thermal sensing system comprising: a conversion device configured to receive one or more electromagnetic signals emitted by a DUT, the conversion device comprising a thermal indicator layer of quantum spin cross-over material configured to change temperature as a function of an electrical and/or magnetic field present at the thermal indicator layer; and an imaging device configured to capture one or more images of the conversion device.

According to one embodiment, the electromagnetic-thermal sensing system further comprises a processing device configured to determine, based on the one or more images, one or more temperature variations in the thermal indicator layer, and to determine one or more energy density values, power density values or entropy values based on the one or more temperature variations.

According to one embodiment, the imaging device is an infrared imaging device.

According to one embodiment, the imaging device is a visible light imaging device, and the conversion device further comprises a functional coating on a side facing the imaging device, the functional coating being configured to change color as a function of temperature.

According to one embodiment, the conversion device is integrated with the imaging device.

According to one embodiment, electromagnetic-thermal sensing system further comprises a further imaging device, configured to capture one or more images of the conversion device, wherein the further imaging device is an IR imaging device.

According to one embodiment, the conversion device further comprises one or more probe or antenna sensors for calibration purposes.

According to one embodiment, the conversion device is patterned with through holes.

According to a further aspect, there is provided a test system comprising the above electromagnetic-thermal sensing system, the electromagnetic-thermal sensing system being configured to sensing electromagnetic emissions from one or more antennas of the DUT.

According to one embodiment, a distance between the DUT and the electromagnetic-thermal sensing system is between 3 and 20 mm.

According to a further aspect, there is provided a method of electromagnetic-thermal sensing comprising: receiving, by a conversion device, one or more electromagnetic signals emitted by a DUT, the conversion device comprising a thermal indicator layer of quantum spin cross-over material configured to change temperature as a function of an electrical and/or magnetic field present at the thermal indicator layer; and capturing one or more images of the conversion device using an imaging device.

According to one embodiment, the method further comprises: determining, by a processing device based on the one or more images, one or more temperature variations in the thermal indicator layer; and determining, by the processing device, one or more energy density values, power density values or entropy values based on the one or more temperature variations.

According to one aspect, there is provided a device configured to measure energy-density, power-density and/or entropy based on measured correlation in a probe array. The device for example comprises a Huygens box comprising probes distributed on its surfaces, and a processing device configured to simultaneously sample signals from a pair of the probes.

According to one embodiment, the correlation is measured by a correlator configured to determine a relation between amplitude and phase of signals received by the probe array.

According to one embodiment, the correlator is configured to perform correlation analysis based on:
modeling and/or measurement of the electromagnetic field emitting from the transmission source; and/or
input data from the probe array.

According to one embodiment, the device further comprises a system for characterizing a transmission source comprising a processing system configured to iteratively characterize, in incremental steps, the transmission field from the probe array towards the source based on the determined amplitude/phase relationship.

According to one embodiment, the processing system is configured to iteratively characterize the transmission field using time-reversal, and/or based on one or more back-propagation algorithms.

According to one embodiment, the probe array comprises absorbers configured to limit emissions from the probe array towards the transmission source.

According to one embodiment, the probe array forms part of a Huygens box, which is for example spherical.

According to one embodiment, the sensor elements of the probe array are spin-wave elements, or any other element sensitive to RF and/or mmWave signals.

According to a further aspect, there is provided a method of measuring entropy, the method comprising measuring correlation in a probe array.

According to one embodiment, the method comprises measuring the correlation by a correlator configured to determine a relation between amplitude and phase of signals received by the probe array.

According to one embodiment, the method comprises characterizing a transmission source, the method comprising:
determining, by the correlator, a relation between amplitude and phase of signals received by a probe array comprising at least two sensor elements; and
iteratively characterizing, by a processing system, in incremental steps, the transmission field from the probe array towards the source based on the determined amplitude/phase relationship.

According to a further aspect, there is provided a system for characterizing a transmission source, the system comprising: a probe array comprising at least two sensor elements; a correlator configured to determine a relation between amplitude and phase of signals received by the probe array; and a processing system configured to iteratively characterize, in incremental steps, the transmission field from the probe array towards the source based on the determined amplitude/phase relationship.

According to one embodiment, the probe array comprises absorbers configured to limit emissions from the probe array towards the transmission source.

According to one embodiment, the probe array forms part of a Huygens box, which is for example spherical or substantially a rectangular parallelepiped shape.

According to one embodiment, the processing system comprises an artificial intelligence module.

According to one embodiment, the correlator is configured to perform correlation analysis based on:
modeling and/or measurement of the electromagnetic field emitting from the transmission source; and/or
input data from the probe array.

According to one embodiment, the processing system is configured to iteratively characterize the transmission field using time-reversal, and/or based on one or more back-propagation algorithms.

According to one embodiment, the sensor elements are spin-wave elements, or any other element sensitive to RF and/or mmWave signals.

According to a further aspect, there is provided a method for characterizing a transmission source, the method comprising: determining, by a correlator, a relation between amplitude and phase of signals received by a probe array comprising at least two sensor elements; and iteratively characterizing, by a processing system, in incremental steps, the transmission field from the probe array towards the source based on the determined amplitude/phase relationship.

According to a further aspect, there is provided a switch matrix system comprising: a plurality of panels, each panel comprising: a plurality N of input/output ports; a plurality M of input/output ports, where M is less than N; and a control circuit configured to synchronize the coupling of one or more selected ones of the N input/output ports to one or more of the M input/output ports; a panel interconnect comprising: a plurality J of input/output ports, each port being coupled to a corresponding one of the M input/output ports of the plurality of panels; a plurality K of input/output ports, where K is less than J; and a further control circuit configured to synchronize the coupling of the one or more selected ones of the N input/output ports of each panel to one or more of the K input/output ports.

According to one embodiment, each of the N input/output ports comprises a connector, each connector for example being suitable for connecting to a sensor such as an antenna.

According to one embodiment, N, M, J and/or K are integers equal to a power of 2.

According to one embodiment, N is equal to at least 16, M is equal to 2 or 4, J is equal to at least 4, and K is equal to 2 or 4.

According to one embodiment, M and K are equal.

According to one embodiment, the synchronization is performed for amplitude and phase.

According to one embodiment, the control circuit of each panel is a programmable circuit, such as an FPGA.

According to one embodiment, the further control circuit is configured to communicate with each of the panel control circuits in order to perform the synchronization.

According to one embodiment, the N input/output ports of each panel is configured to receive a signal at a frequency of up to 30 GHz, and in some embodiments of up to 64 GHz.

According to one embodiment, the switch matrix system further comprising an amplitude adaptation circuit configured to adapt an amplitude of signal present at the K input/output ports, for example based on a control signal received from a driver circuit of a measurement apparatus coupled to the K input/output ports, the amplitude adaptation circuit for example comprising one or more amplifiers and/or attenuators.

According to one embodiment, the K input/output ports are configured to be coupled to input/output ports of an oscilloscope.

According to a further aspect, there is provided a method of coupling a plurality of sensors to a measurement apparatus using the above switch matrix system.

### BRIEF DESCRIPTION OF DRAWINGS

The foregoing features and advantages, as well as others, will be described in detail in the following description of specific embodiments given by way of illustration and not limitation with reference to the accompanying drawings, in which:

FIG. 1 schematically illustrates an electromagnetic-thermal sensing system based on infrared imaging according to an example embodiment of the present disclosure;

FIG. 2 schematically illustrates an electromagnetic-thermal sensing system based on optical imaging according to an example embodiment of the present disclosure;

FIG. 3 schematically illustrates a conversion structure of the sensing systems of FIGS. 1 and 2 hybridized with probe/antenna sensors according to a further example embodiment of the present disclosure;

FIG. 4 schematically illustrates an electromagnetic-optical sensing system based on dual thermal-visual imaging according to an example embodiment of the present disclosure;

FIG. 5 is a flow diagram illustrating an example of operations in a method of DUT OTA testing according to an example embodiment of the present disclosure;

FIG. 6 illustrates an electromagnetic-thermal sensing device with integrated conversion structure according to an example embodiment of the present disclosure;

FIG. 7 illustrates, in plan view, the conversion device of FIG. 1, 2, 4 or 6 hybridized with probe/antenna sensors and a patterned conversion structure according to an example embodiment of the present disclosure;

FIG. 8 schematically illustrates the electromagnetic-thermal sensing system of FIG. 1 showing a device under test in more detail according to an example embodiment of the present disclosure;

FIG. 9 is a graph illustrating energy-density at a distance of 8 mm from a thermal indicator material in the electromagnetic-thermal sensing system of FIG. 8 based on single antenna excitation according to an example embodiment of the present disclosure;

FIG. 10 is a graph illustrating energy-density at a distance of 14 mm from the thermal indicator material in the electromagnetic-thermal sensing system of FIG. 8 based on single antenna excitation according to an example embodiment of the present disclosure;

FIG. 11 is a graph illustrating energy-density at a distance of 10 mm from the thermal indicator material in the electromagnetic-thermal sensing system of FIG. 8 based on simultaneous antenna excitation according to an example embodiment of the present disclosure;

FIG. 12 is a graph illustrating energy-density in the electromagnetic-thermal sensing system of FIG. 8 based on single antenna excitation according to an example embodiment of the present disclosure;

FIG. 13 is a graph illustrating thermal variations in the electromagnetic-thermal sensing system of FIG. 8 based on single antenna excitation according to an example embodiment of the present disclosure;

FIG. 14 is a graph illustrating energy-density squared in the electromagnetic-thermal sensing system of FIG. 8 based on single antenna excitation according to an example embodiment of the present disclosure;

FIG. 15 is a graph illustrating thermal variations in the electromagnetic-thermal sensing system of FIG. 8 based on single antenna excitation as a function of a distance between a DUT and the thermal indicator material according to an example embodiment of the present disclosure;

FIG. 16 illustrates a test environment based on a Huygens box according to an example embodiment of the present disclosure;

FIG. 17 illustrates correlator spherical mapping according to an example embodiment of the present disclosure;

FIG. 18 schematically illustrates a MIMO link with scatterers in the presence of noise including TX and RX adaptive matching according to an example embodiment of the present disclosure;

FIG. 19 illustrates a test system based on a Huygens box according to an example embodiment of the present disclosure;

FIG. 20 schematically illustrates a detection system according to an example embodiment of the present disclosure;

FIG. 21 illustrates a test environment for correlation-based time-reversal calibration solution for probe-array systems using artificial intelligence according to an example embodiment of the present disclosure;

FIG. 22 schematically illustrates modules of a processing device for correlation-based time-reversal calibration solution for probe-array systems using artificial intelligence according to an example embodiment of the present disclosure;

FIG. 23 illustrates operations in a method of correlation-based time-reversal according to an example embodiment of the present disclosure;

FIG. 24 is a graph representing power-density as a function of radiated power in the test environment of FIG. 21 according to an example embodiment of the present disclosure;

FIG. 25 is a graph representing power-density as a function of a distance to a probe array in the test environment of FIG. 21 according to an example embodiment of the present disclosure;

FIG. 26 illustrates a MIMO test solution using smart anechoic chambers with a tunable probe array according to an example embodiment of the present disclosure;

FIG. 27 illustrates the MIMO test solution of FIG. 26 in more detail according to an example embodiment of the present disclosure;

FIG. 28 schematically illustrates a switching system, based on a lego-mosaic approach, for MIMI systems according to an example embodiment of the present disclosure;

FIG. 29 schematically illustrates a test system based on the switching system of FIG. 28 according to an example embodiment of the present disclosure;

FIG. 30 schematically illustrates a test system based on the switching system of FIG. 28 according to a further example embodiment of the present disclosure;

FIG. 31 schematically illustrates a 16-matrix, 32×32 MIMO array switching system according to an example embodiment of the present disclosure;

FIG. 32 illustrates schematically a 32-matrix 64×32 MIMO array switching system to an example embodiment of the present disclosure;

FIG. 33 illustrates a MIMO array switching system comprising two 2-matrix modules according to an example embodiment of the present disclosure;

FIG. 34 illustrates a MIMO array switching system comprising a 16-matrix module according to an example embodiment of the present disclosure;

FIG. 35 is a cross-section view of a re-distribution layer for MIMO systems according to an example embodiment of the present disclosure;

FIG. 36 is a perspective view of the re-distribution layer of FIG. 35 according to an example embodiment of the present disclosure;

FIG. 37 illustrates an equipped robot or person according to an example embodiment; and

FIGS. 38 to 40 are graphs illustrating the influence of the real part and the imaginary part of the permittivity of the thermal indicator material on the sensitivity of the thermal detection.

### DESCRIPTION OF EMBODIMENTS

Like features have been designated by like references in the various figures. In particular, the structural and/or functional features that are common among the various embodiments may have the same references and may dispose identical structural, dimensional and material properties.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following disclosure, unless indicated otherwise, when reference is made to absolute positional qualifiers, such as the terms “front”, “back”, “top”, “bottom”, “left”, “right”, etc., or to relative positional qualifiers, such as the terms “above”, “below”, “higher”, “lower”, etc., or to qualifiers of orientation, such as “horizontal”, “vertical”, etc., reference is made to the orientation shown in the figures.

Unless specified otherwise, the expressions “around”, “approximately”, “substantially” and “in the order of” signify within 10%, and preferably within 5%.

### First Aspect—Electromagnetic-Thermal Sensing for Extracting Energy-Density, Power-Density, or Entropy Values

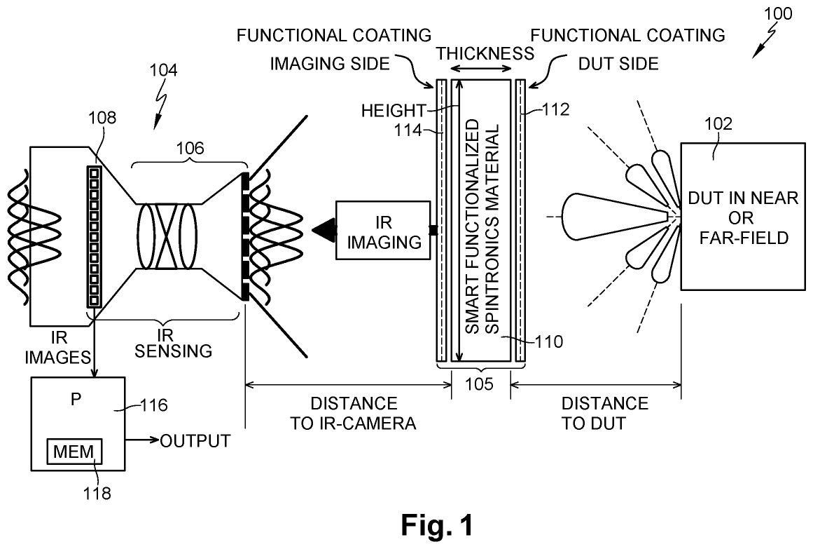

FIG. 1 schematically illustrates an electromagnetic-thermal sensing system 100 based on infrared imaging according to an example embodiment of the present disclosure. The sensing system 100 is for example configured to perform OTA (Over-the-Air) testing of electronic circuits or radiating devices, and in some cases VNF (Very Near Field) testing of circuits and systems. As will be explained in more detail below, the sensing system 100 is based on the functionalization of spintronics indicator material for imaging electromagnetic fields through their thermal signatures.

The system 100 comprises a device under test (DUT IN NEAR OR FAR-FIELD) 102, an infrared (IR) imaging device 104, and a conversion device 105 positioned between the DUT 102 and the IR imaging device.

The DUT 102 for example comprises one or more sources of electromagnetic signals, such as antennas or the like (not illustrated in FIG. 1).

The IR imaging device 104 for example comprises one or more lenses 106, for example integrated within the imaging device 104, for focusing IR light from the conversion device 105 onto an infrared image senor 108. The IR imaging device 104 also comprises an IR image sensor 108 that is sensitive to IR light, and thus suitable for IR sensing (IR SENSING). By IR light, it should for example be understood light with wavelengths equal to or superior to approximately 750 nanometers, and for example in the range of approximately 750 to 1400 nanometers. For example, the IR image sensor 108 comprises an array of pixel circuits, each pixel circuit comprising one or more photodiodes or optoelectronic sensors, which are for example covered by a filter allowing only the infrared wavelengths to pass. Alternatively, other technologies of infrared camera could be employed, such as an IR image sensor based on microbolometers.

The conversion device 105 provides an interface between the DUT 102 and the infrared imaging device 104, and is configured, in particular, to convert electromagnetic signals emitted by the DUT 102 into heat that can be captured by the IR imaging device 104.

The conversion device 105 comprises a thermal indicator layer 110 (SMART FUNCTIONIZED SPINTRONICS MATERIAL) formed of a quantum spin cross-over (SCO) material, also known as a spintronics material. Such materials are known in the art, and are responsive to multi-physics external stimuli such as temperature, pressure, light irradiation, an electromagnetic field, radiation, nuclear decay, soft-X-ray and (de)solvation. In particular, the SCO material is sensitive to the frequencies of electromagnetic signals emitted by the DUT 102, which are for example in the RF or mmWave wavelengths. For example, SCO materials have been shown to be sensitive to a broad frequency spectrum from DC up to RF frequencies and even mmWave frequencies as high as 300 GHz. For example, the DUT 102 is an IoT (Internet of Things) device, or a 5G or 6G communications device.

Examples of spin cross-over materials suitable for

implementing the SCO layer 110 are described in the following publications: Olena Kraieva, Carlos Mario Quintero, Iurii Suleimanov, Edna Hernandez, Denis Lagrange, et al., “High Spatial Resolution Imaging of Transient Thermal Events Using Materials with Thermal Memory”, Small, Wiley-VCH Verlag, 2016, 12 (46), pp.6325-6331, 10.1002/sm11.201601766, hal-01413097; and S. Wane, Q. H.Tran, et al., “Smart Sensing of Vital-Signs: Co-Design of Tunable Quantum-Spin Crossover Materials with Secure Photonics and RF Front-End Modules”, IEEE-MTT-Texas Symposium 2021, the contents of these publications being hereby incorporated by reference. In one embodiment, the SCO material is a material with tan(δ)=0.022, and a thermal conductivity of 0.2 W/(m.K) for a convection coefficient of 15.3 W(m².K) and a relative radiation coefficient equal to 1. The EM-Thermal co-design model is for example meshed using 5.3 Mcells (334×52×104). According to one example, the SCO layer 110 is made of, or comprises, [Fe(HB(1,2,4-triazol-1-yl)₃)₂]bis[hydrotris(1,2,4-triazol-1-yl)borate]Fe(II). This material formula may also comprise additional H₂O compounds.

The conversion device 105 is for example substantially planar or disc-shaped, and is for example arranged in a plane that is substantially perpendicular to an axis passing through an emission source of the DUT 102 and an optical axis of the IR imaging device 104.

The SCO layer 110 for example has a thickness (THICKNESS) in the range of 1 micrometer to 5 mm, and preferably in the range 0.01 mm to 1 mm. An advantage of providing the SCO layer 110 with a relative low thickness of less the 1 mm, and for example less than 0.5 mm, is that the losses as the energy passes through the layer 110 can be relatively low, leading to a higher signal on the imager side. The SCO layer 110 for example has a width (not represented in FIG. 1, and corresponding to a direction perpendicular to the plane shown in the figure), and/or height (HEIGHT) of between 10 and 100 mm, and preferably of between 20 and 50 mm.

The conversion device 105 is for example in the far field, near field, or very near field of the DUT 102. In some embodiments, the layer 110 of the conversion device 105 is spaced from the DUT 102 by a distance (DISTANCE TO DUT) in the order of a wavelength at the frequency to be detected, and thus at about 10 mm at 30 GHz. For example, the distance between the layer 110 and the DUT 102 is between 0.5 λ and 5 λ, where λ is the wavelength. In some embodiments, the layer 110 of the conversion device 105 is spaced from the imaging device 104, such as from a first lens of the imaging device 104, by a spacing (DISTANCE to IR-CAMERA) that is a function of the resolution and the desired signal-to-noise-ratio.

In some embodiments, the layer 110 is a smart functionalized spintronics material, the conversion device 105 comprising function coatings 112 and/or 114 on the DUT 102 or imager 104 side.

For example, the functional coating 112 on the DUT side is an insulating layer, for example formed of a polymer of between 10 and 200 μm in thickness, that is configured to permit electromagnetic signals to pass through, while blocking to some extent heat originating from the DUT 102 from reaching the SCO layer 110. Indeed, direct heating of the SCO layer 110 caused by heat emitted by the DUT 102 adds unwanted noise to the thermal output of the SCO layer 110.

The functional coating 114 on the imaging device side is for example a material that increases the sensitivity of the thermal detection by the IR imaging device 104. For example, the functional coating 114 is formed of a polymer of between 10 and 200 μm in thickness comprising magnetic particles or the like, configured to bring heat generated inside the layer 110 to the exterior surface of the layer 114 facing the imaging device 104, and thereby improving image detection by the imaging device 104.

It has been observed by the inventor that there is a direct link between thermal variations in the SCO material of layer 110 and the square of the electric and magnetic fields present at the layer 110. Indeed, a few tens of dBm input power emitted by the DUT 102 results in a few degrees of dynamic heating within the SCO layer 110. Field amplitudes can be obtained by the following relations.

For Electric-Field as primary sensing field:

|E|=X_{EM-Thermal}^{E} \sqrt{ΔT_{Averaged}}

where |E| is the magnitude of the electric field, X_{EM-Thermal}^{E} is an electric field to temperature conversion coefficient, and ΔT_{Averaged} is the temperature variation in the SCO material resulting.

For Magnetic-Field as primary sensing field:

|H|=X_{EM-Thermal}^{E} \sqrt{ΔT_{Averaged}}

where |H| is the magnitude of the magnetic field, X_{EM-Thermal}^{H} is a magnetic field to temperature conversion coefficient, and ΔT_{Avereged} is the temperature variation in the SCO material, averaged in time and/or space. For example, in some embodiments, the pixel value of each pixel of the IR image is averaged over several successive frames in order to generate the value ΔT_{Averaged}. Additionally or alternatively, the pixel values of neighboring pixels in the IR image are averaged in order to generate the value ΔT_{Averaged} for a group of pixels. Furthermore, in order to extract the temperature difference ΔT, the ambient temperature is for example subtracted from each pixel value. For relatively stable environments, for example in controlled settings, the ambient temperature can be extracted from the IR images, and can be considered as uniform across the conversion device 105. For this, the IR camera is for example configured to capture one or more zones outside of the conversion device 105, and such zones can be considered to be at the ambient temperature. For unstable environments, the ambient temperature is for example determined for each pixel by capturing a reference IR image with the DUT deactivated, and then capturing a further IR image with the DUT activated and emitting the electromagnetic signals to be detected.

The conversion coefficients X_{EM-Thermal}^{E} and X_{EM-Thermal}^{H} depend on the heat transfer coefficient, the heat capacity, the density of the SCO material, and the frequency of the detected signal.

From the above equations, the power density IsPDI can be deduced in the form:

|sPD|=∝ X_{EM-Thermal}^{E} X_{EM-Thermal}^{H}ΔT_{Averaged}

In some embodiments, the temperature change ΔT_{Averaged} is a spatial average among a group of pixels of the IR image, and the power density |sPD| is thus also a spatial average.

The electromagnetic-thermal sensing system 100 further comprises, for example, a processing device (P) 116 coupled to an output of the IR image sensor 108, and configured to receive IR images (IR IMAGES) from the image sensor 108. The processing device 116 for example comprises a memory (MEM) 118 configured to store each of the conversion coefficients X_{EM-Thermal}^{E} and X_{EM-Thermal}^{E}, or a combined conversion coefficient X_{EM-Thermal}^{E+H} equal to the product X_{EM-Thermal}^{E}X_{EM-Thermal}^{H}. The processing device 116 for example comprises one or more processing units under control of instructions stored in the memory, and/or a hardware circuit for performing image processing, such as an FPGA (Field Programmable Gate Array) or ASIC (Application-Specific Integrated Circuit), including SoC (System on a Chip) solutions. The processing device 116 is for example configured to process pixel data of the IR image and to generate, based on the pixel data and on the conversion coefficients or combined conversion coefficient, one or more output values (OUTPUT) representing energy density and/or power density values in relation with the electric and magnetic fields emitted by the DUT 102, based on the above equations.

In addition to, or rather than, calculating power or energy density values, entropy values can be generated. The extraction of energy density, power density and entropy is described for example in more detail in the publication by S. Wane et al. entitled “Energy-Geometry-Entropy Bounds aware Analysis of Stochastic Field-Field Correlations for Emerging Wireless Communication Technologies”, URSI General Assembly Commission, New Concepts in Wireless Communications, Montreal 2017), the contents of this publication being hereby incorporated by reference.

An advantage of the sensing system 100 of FIG. 1 is that testing at nano-scale resolutions can be achieved, at far reduced cost when compared to prior art solutions.

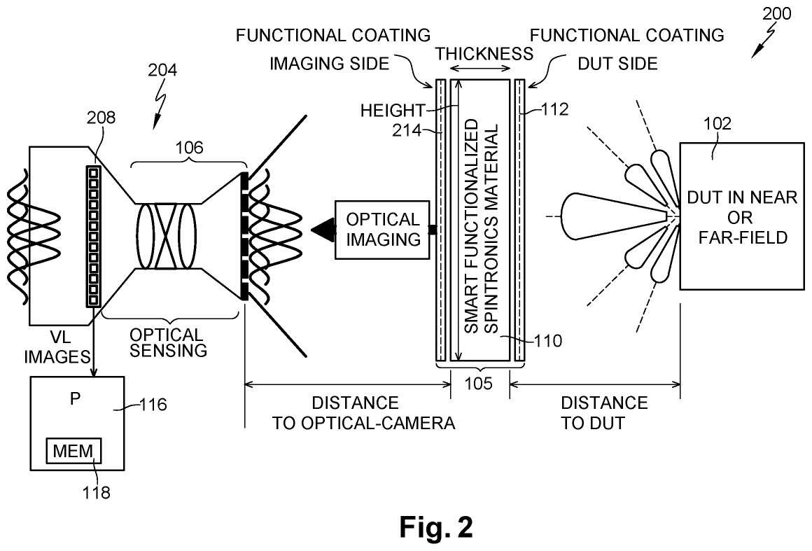

FIG. 2 schematically illustrates an electromagnetic-optical sensing system 200 based on optical imaging according to an example embodiment of the present disclosure. The sensing system 200 is similar to the sensing system 100 of FIG. 1, and like features are labelled with like reference numerals and will not be described again in detail.

In the sensing system 200, the IR imaging device 104 is replaced by an optical imaging device 204 configured to capture visible light images using an image sensor 208, which is for example a CMOS image sensor. By visible light, it should for example be understood light with wavelengths ranging from approximately 350 nanometers to approximately 750 nanometers. The visual image sensor 208 for example comprises one or a plurality of photodiodes or optoelectronic sensors. For example, the visual image sensor comprises an array of pixel circuits, each pixel circuit comprising one or more photodiodes or optoelectronic sensors. In the case that the visual imaging device 204 is a color camera, at least some of the photodiodes are for example covered by a color filter.

In this embodiment, the conversion device 105 is further configured to convert temperature variations into color variations. For example, the SCO layer 110 is coated, on the imager side, with a functional coating that is configured to have a color that varies locally as a function of the temperature variations of the SCO layer 110. Such color-changing coatings responsive to temperature variations are known in the art. Examples of types of materials that could be used include photonic materials, fluorescent materials, or the like, Nano particles functionalized in polymers, graphene, etc.

Operation of the sensing system 200 is similar to that the sensing system 100 of FIG. 1. However, in the system 200, the processing device 116 is for example configured to extract the temperature variation of each pixel as a function of its color, for example based on RGB color channels, rather than being based on a single IR pixel value.

An advantage of the sensing system 200 of FIG. 2 is that the imaging device can be visible light camera, rather than a more-costly IR camera.

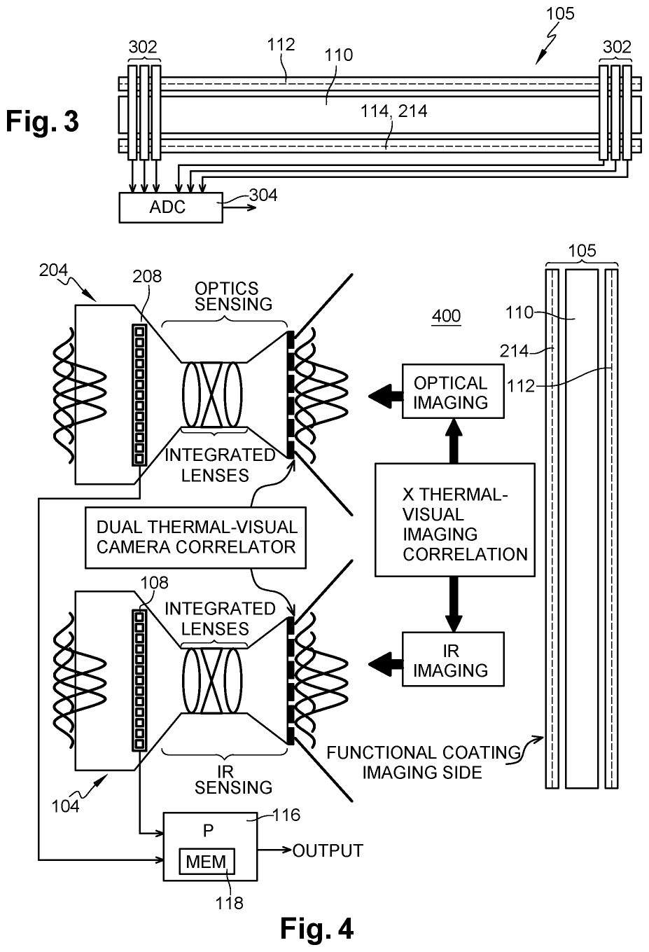

FIG. 3 schematically illustrates the conversion device 105 of the sensing systems 100, 200 of FIGS. 1 and 2 according to an embodiment in which it is hybridized with probe/antenna sensors 302. For example, the probe/antenna sensors 302 are provided close to the edges of the devices. The sensors 302 are for example sensitive to RF and/or mmWave wavelengths, depending on the frequencies emitted by the DUT 102, and are for example sensitive to similar frequencies to those of the SCO layer 110.

The probe/antenna sensors 302 for example comprise spin-wave or spintronics-based magnetic sensors. Sensors based on Spintronics are for example described in more detail in the publications: Q. H. Tran, S. Wane, et al., “Toward Co-Design of Spin-Wave Sensors with RFIC Building Blocks for Emerging Technologies”, 2018 2nd URSI Atlantic Radio Science Meeting (AT-RASC)”; P. P. Freitas, et al., “Spintronic Sensors” Proc. of the IEEE 104 (10)1894 (2016)DOI: 10.1109/JPROC.2016.2578303; in the European patent application published as EP3208627 by F. TERKI et al. entitled “Measurement system and method for characterizing at least one single magnetic object”, and in the International Patent application entitled “Spin-Wave based Magnetic and/or Electro-magnetic Field Sensing Device for DC, RF and Millimeter-Wave Applications” published as WO2021/094587, the contents of each of these application being hereby incorporated by reference. Alternatively, the sensors 302 could be antennas configured to receive electromagnetic signals. The probe/antenna sensors 302 are for example configured to provide output measurements to an ADC, which is in turn configured to provide digital readings to the processing device 116 (not illustrated in FIG. 3). These readings for example permit a calibration of the IR and/or color values captured by the imaging device 104 or 204. In particular, the probe/antenna sensors 302 provide an indication of the electric and/or magnetic field strength present at the conversion device 105.

FIG. 4 schematically illustrates an electromagnetic-optical sensing system 400 based on dual thermal-visual imaging according to an example embodiment of the present disclosure. The system 400 is similar to the systems 100 and 200 of FIGS. 1 and 2, except that it comprises both the IR imaging device 104, and the visible light imaging device 204, configured to image the same conversion device 105 (for ease of illustration, the DUT 102 is not illustrated in FIG. 4, but the same DUT will be present in the system 400, like in the embodiments of FIGS. 1 and 2). The conversion device 105 for example comprises the function layer 214 configured to change color in response to temperature changes such that the color variations can be captured by the visible imaging device 204, and this layer 214 also for example has local temperatures differences provides the thermal variations that can be captured by the IR imaging device 104. The conversion device 105 of FIG. 4 optionally includes the probe/antenna sensors 302 of FIG. 3 (not illustrated in FIG. 4).

The IR and visual imaging devices 104, 204 of the system 400 are for example arranged as close as possible to each other to provide frames representing the scene from two view points that are relatively similar. The optical axes of the IR and visual imaging devices are for example aligned so as to be substantially parallel to each other, or to converge to a common point on the conversion device 105.

The output signals of the image sensors 108, 208 of the imaging devices 104, 204 are for example both provided to the processing device 116, which in this embodiment is configured to generate power density, energy density values, or entropy values, based on the pixel values of both the IR and visible light images. In some embodiments, the visible light imaging device 204 has a greater resolution than the IR imaging device. For example, the visible light imaging device 204 is a 4K imaging device, also known has an ultra HD (high definition) device, and the processing device 116 is configured to convert the resolution of the visible images to the same resolution as the IR images prior to generating the are power density values, energy density values, or entropy values. In some embodiments, the use of both IR images and visible light images permits the resolution of the resulting output images to be improved and also allows a calibration or correction of the readings with respect to each other. Indeed, each of the imaging devices 104, 204 provide temperature information concerning the conversion layer 105 based on a different technique, and thus combining the readings permits the precision to be improved.

In some embodiments, the processing device 116 is configured to generate thermal-visual correlations between pixel values generated by the imaging devices 104, 204, such correlation values leading to greater precision. Techniques for aligning thermal and IR images are for example described in more detail in the PCT patent application having application number PCT/EP2021/064578 filed on 31 May 2021, the contents of which is hereby incorporated by reference.

The attributes of the proposed Dual Thermal-Visual Camera Correlator solutions include the following technological differentiators:
Use of functionalized spintronics materials for tailored sensitivity of hybrid Electromagnetic-Thermal conversions. The tailoring of the sensitivity uses broadband extended Kramers-Kronig relations derived in time-domain. The tailoring process is based on 3D patterning of SCO thermal indicator materials designed to simultaneously meet RF/mmWave Electromagnetic and Thermal requirements for high sensitivity with reduced invasiveness. The functionalization of the SCO materials uses in-house Electromagnetic-Thermal co-design optimizations.
Use of correlation technologies combined with advanced interferometric synchronized sampling for accurate extraction of vectorial power and energy density metrics in time and frequency domains.
Use of high resolution IR-cameras for calibrated Electromagnetic-Thermal conversions taking advantage of advanced FDSOI technology platforms toward co-integration of SCO materials with Front-End-Modules (FEMs) for pushing thermodynamic sensitivities to their ultimate performance limits. The co-integration of SCO materials with smart FEMs will foster new avenues for replacing conventional IR-cameras with low-cost visible cameras considering fluorescent functionalization processes.

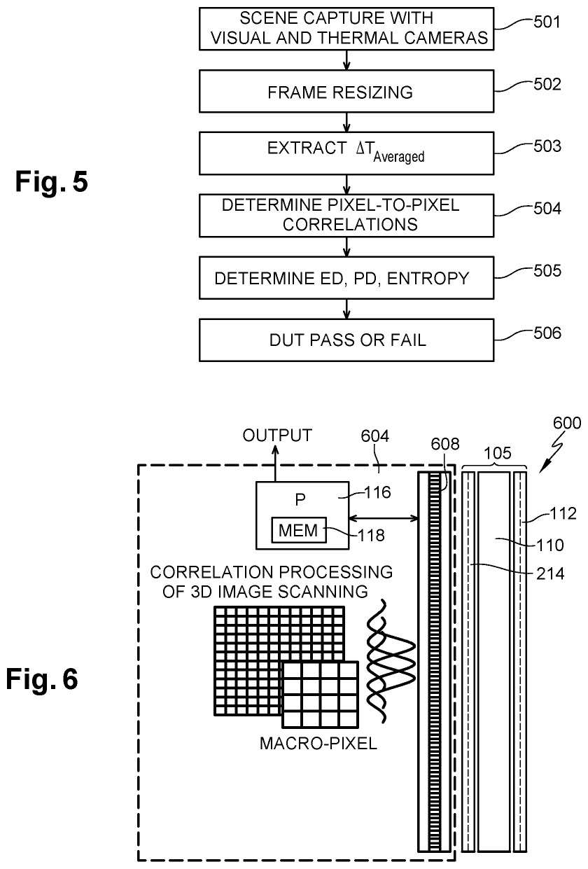

FIG. 5 is a flow diagram illustrating an example of operations in a method of DUT OTA testing according to an example embodiment of the present disclosure. The method of FIG. 5 is for example performed by the sensing system 400 of FIG. 4, under control of the processing device 116.

In an operation 501 (SCENE CAPTURE WITH VISUAL AND THERMAL CAMERAS), a scene capture is performed using the IR and visual imaging devices 104, 204.

The visual image sensor 208 is for example configured to capture a scene, including the conversion device 105, during a capture period. The visual image sensor 208 generates one or more visual frames during the capture period. Capturing a plurality of frames permits time averaging of the pixel values to be performed. The visual frames are represented by pixels P[i,j], where [i,j] represents the pixel location in frame. The pixels P[i,j] are for example indexed as a function of their relative position in each frame along two virtual perpendicular axes. Each pixel P[i,j] is for example composed of a single component, for example in the case of greyscale pixels, or of several components, for example in the case of color pixels. For example, in the case of color pixels, each pixel for example comprises red, green, and/or blue components, and/or other components, depending on the encoding scheme.

The IR image sensor 108 is for example configured to capture the scene, including the conversion device 105, during the same capture period as the visual image sensor 208. In over words, the image capture times of the visual and IR imaging devices 104, 204 are for example synchronized with each other. The IR image sensor 108 generates one or more thermal frames during the capture period. Capturing a plurality of frames permits time averaging of the pixel values to be performed. The thermal frames are represented by pixels P[k,l], where [k,l] represents the pixel location in frame. The pixels P[k,l] are for example indexed as a function of their relative position in each frame along two virtual perpendicular axes. Each pixel P[k,l] is for example composed of a single component, for example in the case of greyscale pixels, or of several components, for example in the case of color pixels. For example, in the case of color pixels, the colors are generated during a pre-processing operation of the pixels at the output of the thermal image sensor, for example in order to aid the visualization of the thermal information. In this case, each pixel for example comprises red, green, and/or blue components, or other components, depending on the encoding scheme.

In an operation 502 (FRAME RESIZING), the processing device 116 is optionally configured to resize the visual frame and/or the thermal frame, such that they have a same common size. This step is optional and may facilitate the signal processing, for example in the case that the resolution of the visual frames is greater than that of the thermal frames.

In an operation 503 (EXTRACT ΔT_{Averaged}), average temperature variations ΔT_{Averaged} are for example extracted for each of the visual and thermal frames. For example, this is achieved by subtracting an ambient temperature from each pixel value, such that the remainder is equal to the temperature variation, as explained above in relation with FIG. 1.

In an operation 504 (DETERMINING PIXEL-TO-PIXEL CORRELATIONS), optionally a plurality of pixel-to-pixel correlation values are for example determined between first pixel values of pixels P[i,j] of one of the visual frames and first pixel values of pixels P[k,l] of a corresponding one of the thermal frames. The term “value” of pixel corresponds similarly to an intensity and for example to an intensity corresponding to each color contained in subpixels of the pixels, such as red, green or blue.

In an example, the various pixel intensities are transformed to be represented by gaussian curves.

The pixel-to-pixel correlations may be obtained by auto-correlations

$${nAC}_{{I}_{{S}_{1}} {I}_{{S}_{1}}} ( τ )$$

and/or cross-correlations

$${nCC}_{{I}_{{S}_{1}} {I}_{S 2}} ( τ ) ,$$

based for example on the following normalized equations (equations 1 and 2):

$${nAC}_{{I}_{{S}_{1}} {I}_{{S}_{1}}} ( τ ) = \frac{{\int }_{-}^{+} {I}_{{S}_{1}} ( t ) {I}_{{S}_{1}} ( t + τ ) dt}{\sqrt{{\int }_{-}^{+} {\vert {I}_{{S}_{1}} ( t ) \vert}^{2} dt {\int }_{-}^{+} {\vert {I}_{{S}_{1}} ( t ) \vert}^{2} dt}} \qquad [ Math 1 ]$$

where τ is the time lag, which will also be referred to herein as the correlation displacement parameter, and I_{Sl} is a matrix of pixel values of an image region or entire frame for which the auto-correlation is to be determined.

$${nCC}_{{I}_{{S}_{1}} {I}_{S 2}} ( τ ) = \frac{{\int }_{-}^{+} {I}_{{S}_{1}} ( t ) {I}_{{S}_{2}} ( t + τ ) dt}{\sqrt{{\int }_{-}^{+} {\vert {I}_{{S}_{1}} ( t ) \vert}^{2} dt {\int }_{-}^{+} {\vert {I}_{{S}_{2}} ( t ) \vert}^{2} dt}} \qquad [ Math 2 ]$$

where I_{S}_{₁} is a matrix of pixel values of an image region or entire frame of one of the frames, for example one of the visual frames, and I_{S}_{₂} is a matrix of pixel values of an image region or entire frame of the other frames, for example one of the thermal frames, the correlation for example corresponding to an average value based on corresponding pixel-to-pixel correlations, for example generated based on each of the corresponding pixels P[i,j] and P[k,l].

In an operation 505 (DETERMINE ED, PD, ENTROPY), one or more of an energy density, power density, and entropy are determined based on the extracted average temperature variations ΔT_{Averaged} generated in operation 503, and/or based on the pixel-to-pixel correlations generated in operation 504.

The conventional definition of the physical entropy S of a system with a particular macrostate—e.g., energy, composition, volume, (U,N,V)—in statistical physics and that of information H(z), can be linked by the following equation:

H(z)=S(U, N, V)/kln(2)=−ΣₛP_{z} (s) log₂ P_{z} (s) (1)

where k is the Boltzmann constant.

The energy U is composed of Electric and Magnetic energies. The Volume V is composed of meshed pixels. Correlations functions are extracted at pixel level.

Proposed Entropy Measurement solutions enable efficient combination of Information-Signal Theory (IT) & Physical Information Theory (PT) into a unified approach: Shannon's entropy can be directly related to Boltzmann's entropy for assessing the quality of RF wireless systems: e.g., SNR, EVM, Channel-Capacity, can be accurately extracted.

$$I ( X , Y ) = {log}_{2} det [ I + \frac{1}{{σ}_{v}^{2}} {HR}_{v}^{- 1} {H}^{H} {R}_{X} ] \\ I ( X , Y ) = {log}_{2} det [ I + \frac{1}{{σ}_{v}^{2}} {HR}_{v}^{- 1} {H}^{H} {R}_{X} ]$$

where I(X,Y) is related to Differential Entropy (Maximization), H is the Channel Transfer Matrix, and each of Rᵥ and R_{X} is a Correlation Matrix:

$${R}_{v} = \frac{1}{{σ}_{v}^{2}} E [ {vv}^{H} ] \\ {R}_{X} = E [ {XX}^{H} ]$$

The Shannon-McMillan-Breiman theorem provides a formal bridge between the Boltzmann entropy and the Shannon entropy. In equation (1), the average information in a set of messages associated to probabilities Pz(s) map onto the ensemble of the microstates of the physical system. The variable z is a label for the set of possible messages and the probability over this set, s is a particular value from the set. Equation (1) is valid in the case of non-equilibrium systems, for a well-defined ensemble probability distribution, Pz(s), several conceptual difficulties arises from the physical interpretation of system complexity in link with equilibrium entropy.

The energy density can be written as the sum of electric and magnetic energy densities [R. F. Harrington, Time-Harmonic Electromagnetic Fields. New York: McGraw-Hill, 196.]:

$$W ( ρ ) = {W}_{E} ( ρ ) + {W}_{H} ( ρ ) \\ {W}_{E} ( ρ ) = \frac{ε}{2} {\vert E ( ρ ) \vert}^{2} and {W}_{H} ( ρ ) = \frac{μ}{2} {\vert H ( ρ ) \vert}^{2}$$

The correlation function of the electric or magnetic field is defined as:

$${C}_{X}^{FF} ≡ \frac{〈 X ( {ρ}_{1} ) . {X}^{*} ( {ρ}_{2} ) 〉}{〈 {\vert X ( {ρ}_{1} ) \vert}^{2} 〉 〈 {\vert X ( {ρ}_{2} ) \vert}^{2} 〉}$$

where [symbol] X [symbol] refers to ensemble average (expectation) applied to stochastic variable X and * stands for complex conjugate.

The correlation function of the electric energy density can be deduced as:

$${C}_{{W}_{E}}^{FF} ≡ \frac{〈 [ {W}_{E} ( {ρ}_{1} ) - 〈 {W}_{E} ( {ρ}_{1} ) 〉 ] [ {W}_{E} ( {ρ}_{2} ) - 〈 {W}_{E} ( {ρ}_{2} ) 〉 ] 〉}{\sqrt{{〈 [ {W}_{E} ( {ρ}_{1} ) - 〈 {W}_{E} ( {ρ}_{1} ) 〉 ]}^{2} 〉 {〈 [ {W}_{E} ( {ρ}_{2} ) - 〈 {W}_{E} ( {ρ}_{2} ) 〉 ]}^{2} 〉}}$$

$${C}_{{W}_{H}}^{FF} ≡ \frac{〈 [ {W}_{H} ( {ρ}_{1} ) - 〈 {W}_{H} ( {ρ}_{1} ) 〉 ] [ {W}_{H} ( {ρ}_{2} ) - 〈 {W}_{H} ( {ρ}_{2} ) 〉 ] 〉}{\sqrt{{〈 [ {W}_{H} ( {ρ}_{1} ) - 〈 {W}_{H} ( {ρ}_{1} ) 〉 ]}^{2} 〉 {〈 [ {W}_{H} ( {ρ}_{2} ) - 〈 {W}_{H} ( {ρ}_{2} ) 〉 ]}^{2} 〉}}$$

For stationary stochastic signals, the spatial correlation functions for the total field Xₜ exhibit a SinC(kρ) law.

C_{X}_{ₜ}^{FF} (ρ)=∝ SinC(kρ)

The spatial correlation functions of the transverse components Xₜ can be expressed as:

$${C}_{{X}_{t}}^{FF} ( ρ ) = \frac{3}{2} \{ Sin C ( k ρ ) - \frac{1}{{( k ρ )}^{2}} [ Sin C ( k ρ ) - κ Sin C ( \frac{k ρ}{2} ) ] \}$$

where it can be established that

$$κ = cos ( \frac{k ρ}{2} ) .$$

The SinC(kρ) law can be implemented using advanced signal processing convolutional accelerators implementing broadband expansions:

$$Sin C ( k ρ ) = \frac{Sin ( k ρ )}{k ρ} = {\sum }_{n = 1}^{n = ∞} {( - 1 )}^{n} \frac{{( k ρ )}^{2 n}}{( 2 n + 1 ) !}$$

$$Sin C ( k ρ ) = {\prod }_{k = 1}^{∞} cos ( \frac{k ρ}{{2}^{k}} )$$

In an operation 506 (DUT PASS OR FAIL), the DUT 102 is for example evaluated based on one or more of the energy density, power density or entropy values generated in operation 505. For example, in some cases, the DUT 102 may fail the OTA test if the energy density, power density or entropy of the signal emitted by any antenna of the DUT 102 is outside of a desired range, indicating for example that the antenna is faulty and thus not emitting sufficient signal, or is over emitting, which could result in harmful levels of radiation. In some embodiments, the processing device 116 generates an output signal indicating when the DUT passes or fails, and this output signal is used to control one or more robotic systems in order to selectively bin the DUT 102 as a function of the pass or fail decision. Of course, the binning of the DUT 102 based on the energy density, power density or entropy is merely one example, and in alternative embodiments other actions could be taken in response to the determined output values.

While FIG. 5 illustrate the operation of the sensing system 400 of FIG. 4, it will be apparent to those skilled in the art how this operation could be adapted for the sensing systems of FIG. 1 or 2.

FIG. 6 illustrates an electromagnetic-thermal sensing device 600 with integrated conversion device according to an example embodiment of the present disclosure.

The conversion device is for example the conversion device 105 of FIG. 2 comprising at least the function coating 214, which is sandwiched between the SCO layer 110 of the conversion device 105 and the imaging device 604. The conversion device 105 of FIG. 6 optionally includes the probe/antenna sensors 302 of FIG. 3 (not illustrated in FIG. 6). In particular, the device 600 of FIG. 6 comprises an imaging device 604 having a visible light image sensor 608, and the conversion device 105 integrated with the imaging device 604, such that the image sensor 608 receives light generated by the functional coating 214.

In the embodiment of FIG. 6, the dimensions of the conversion device 105 are substantially the same as those of the image sensor 608 in the plane perpendicular to the optical axis, and the conversion device 105 and imaging device 604 are aligned such that all, or substantially all, of the pixels of the image sensor 608 are covered by the conversion layer 105.

The imaging device 604 for example comprises the processing device 116 configured to process images captured by the image sensor 608, such that the imaging device 604 is capable of outputting energy density, power density and/or entropy values directly as an output signal (OUTPUT), based for example on correlation processing (CORRELATION PROCESSING OF 3D IMAGE SCANNING), which in some embodiments is based on macro-pixel processing.

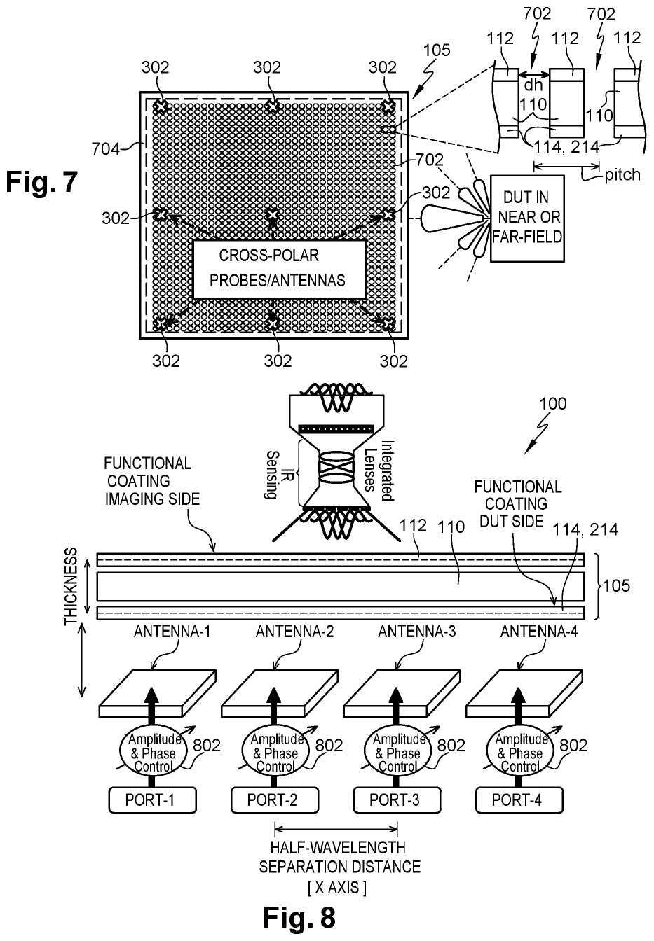

FIG. 7 illustrates, in plan view, the conversion device 105 of FIG. 1, 2, 4 or 6 according to a further example embodiment in which it is for example hybridized with probe/antenna sensors 302, which are for example cross-polar probes/antennas (CROSS-POLAR PROBES/ANTENNAS) in the example of FIG. 7. Furthermore, the SCO layer 110 is for example patterned with through holes 702. Two of the holes 702 are illustrated in more detail in a cross-section cutout of FIG. 7. As illustrated by this cross-section, the functional coatings 112, 114, 214, if present, also for example have the same hole pattern, such that the holes 702 are through holes passing entirely through the conversion device 105. The holes 702 for example each have a diameter dh of between 100 μm and 1 mm, and a pitch that is for example equally to between 1 and 4 times the hole diameter dh, and for example substantially equal to twice the hole diameter dh. In some embodiments, the conversion device 105 comprises a thermal convection shield 704 surrounding the SCO layer 110 on all edges. The thermal convection shield 704 is for example of Polyethylene(PE)-Foil material or a similar composition.

FIG. 8 schematically illustrates the electromagnetic-thermal sensing system 100 of FIG. 1 showing the device under test 102 in more detail according to an example embodiment of the present disclosure. In the example of FIG. 8, the DUT 102 is a device comprises four antennas ANTENNA-1, ANTENNA-2, ANTENNA-3 and ANTENNA-4, which are for example patch antennas. Each antenna is driven via a corresponding port PORT-1, PORT-2, PORT-3 and PORT-4, with an amplitude and phase control circuit 802 coupled between each port and each antenna, permitting adjustment of the amplitude and/or phase of the signal to be transmitted. The antennas ANTENNA-1 to ANTENNA-4 are for example arranged in a single row along an X axis, with adjacent antennas being separated by a half-wavelength (HALF-WAVELENGTH SEPARATION DISTANCE [X AXIS]) of the transmission frequency to be transmitted.

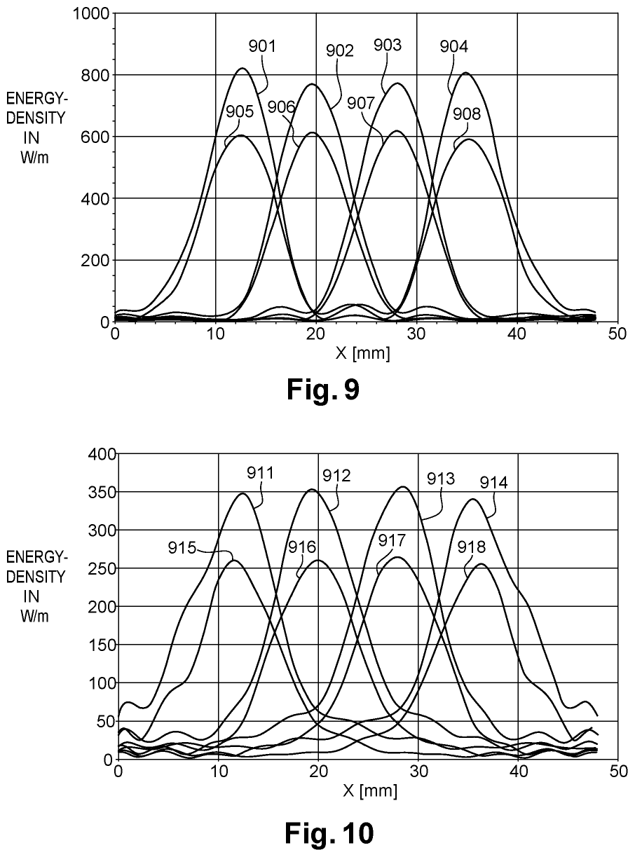

FIGS. 9 to 15 are graphs illustrating results obtained based on imaging the DUT 102 of FIG. 8.

FIG. 9 is a graph illustrating energy-density as a function of X position, with the thermal indicator material layer 110 at a distance of 8 mm from the DUT 102 in the electromagnetic-thermal sensing system of FIG. 8 based on single antenna excitation. Excitation was at 26 GHz, and energy density was extracted at this frequency. Curves 901, 902, 903 and 904 correspond respectively to excitation of the antennas ANTENNA-1 to ANTENNA-4 of FIG. 8, based on an SCO layer 110 of 0.5 mm in thickness. Curves 905, 906, 907 and 908 correspond respectively to excitation of the antennas ANTENNA-1 to ANTENNA-4 of FIG. 8, based on an SCO layer 110 of 0.9 mm in thickness. The curves 901 to 908 were generated with a same transmission power to each antenna, and it can be seen that a lower thickness of the SCO layer 110 leads to a higher detection sensitivity.

FIG. 10 is a graph illustrating energy-density as a function of X position, with the thermal indicator material layer 110 at a distance of 14 mm from the DUT 102 in the electromagnetic-thermal sensing system of FIG. 8 based on single antenna excitation. Excitation was at 26 GHz, and energy density was extracted at this frequency. Curves 1001, 1002, 1003 and 1004 correspond respectively to excitation of the antennas ANTENNA-1 to ANTENNA-4 of FIG. 8, based on an SCO layer 110 of 0.5 mm in thickness. Curves 1005, 1006, 1007 and 1008 correspond respectively to excitation of the antennas ANTENNA-1 to ANTENNA-4 of FIG. 8, based on an SCO layer 110 of 0.9 mm in thickness. The curves 1001 to 1008 were generated with a same transmission power to each antenna, and it can be seen that a lower thickness of the SCO layer 110 leads to a higher detection sensitivity. Energy densities in FIG. 9B are less than half the values of FIG. 9 due to the increased distance of the thermal indicator material layer 110 from the DUT 102.

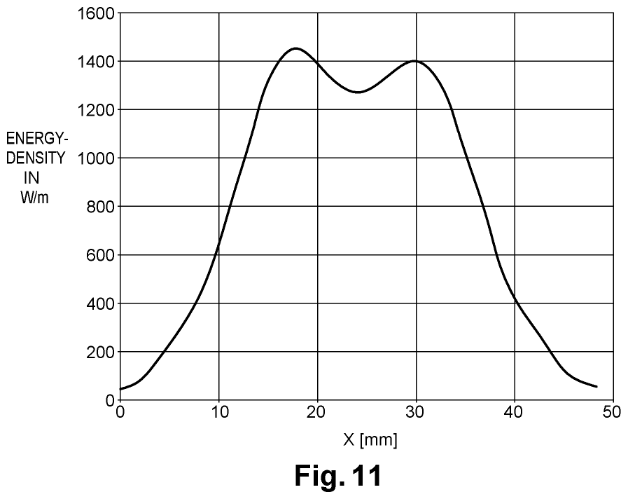

FIG. 11 is a graph illustrating energy-density as a function of X position, with the thermal indicator material layer 110 at a distance of 10 mm from the DUT 102 in the electromagnetic-thermal sensing system of FIG. 8 based on simultaneous antenna excitation according to an example embodiment of the present disclosure. Excitation was at 26 GHz, and energy density was extracted at this frequency. The excitation power was for example 23 dBm per port.

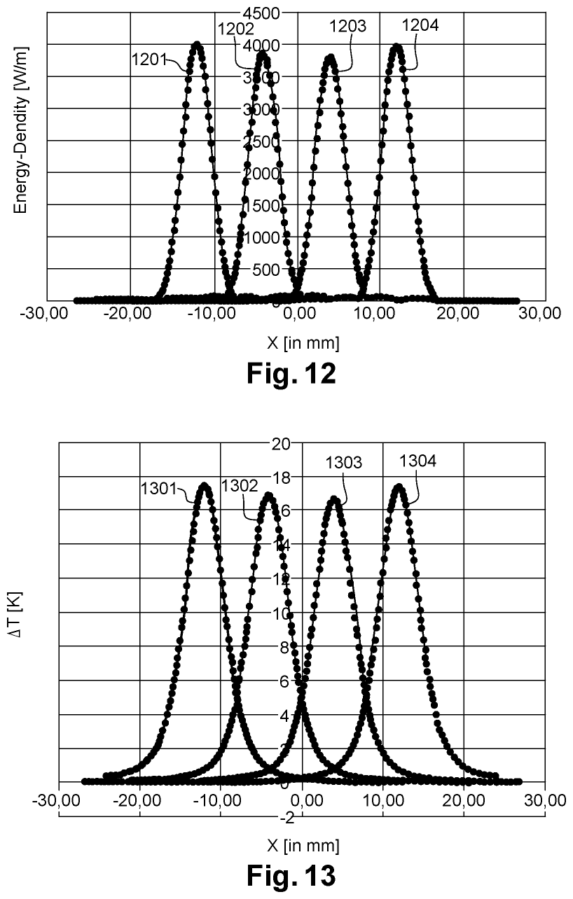

FIGS. 12 to 15 illustrate energy-density and thermal

measurements with the thermal indicator material layer 110 at a distance of 3 mm from the DUT 102 in the electromagnetic-thermal sensing system of FIG. 8 based on single antenna excitation according to an example embodiment of the present disclosure. Excitation was at 26 GHz, and energy density was extracted at this frequency.

FIG. 12 illustrates energy-density [W/m] as a function of X position, and the curves 1201, 1202, 1203 and 1204 respectively correspond to excitation of the antennas ANTENNA-1, ANTENNA-2, ANTENNA-3 and ANTENNA-4.

FIG. 13 illustrates temperature variation ΔT [K] as a function of X position, and the curves 1301, 1302, 1303 and 1204 respectively correspond to excitation of the antennas ANTENNA-1, ANTENNA-2, ANTENNA-3 and ANTENNA-4.

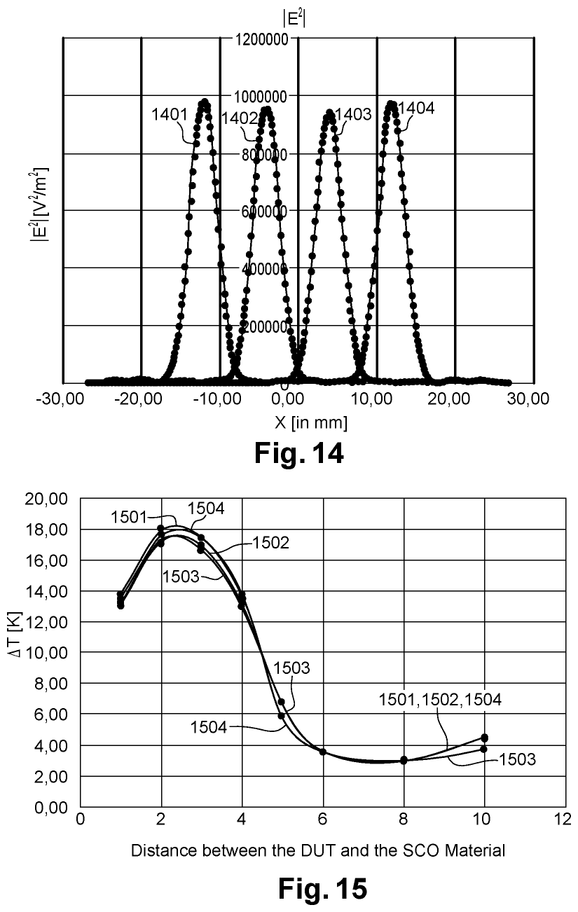

FIG. 14 illustrates energy-density squared |E|² [V²/m²] as a function of X position, and the curves 1401, 1402, 1403 and 1404 respectively correspond to excitation of the antennas ANTENNA-1, ANTENNA-2, ANTENNA-3 and ANTENNA-4.

FIG. 15 illustrates temperature variation ΔT [K] as a function of the distance between the thermal indicator material layer 110 and the DUT 102. Curves 1501, 1502, 1503 and 1504 respectively correspond to excitation of the antennas ANTENNA-1, ANTENNA-2, ANTENNA-3 and ANTENNA-4. It can be seen that the peak sensitivity in temperature is achieved at a distance from the DUT of around 3 mm, but that sensitivity remains reasonable and relatively constant from 6 mm to 10 mm.

FIGS. 38 to 40 are graphs illustrating the influence of the real part and the imaginary part of the permittivity of the thermal indicator material on the sensitivity of the thermal detection.

FIG. 38 illustrates in particular the imaginary part Tan(delta) against the real part Real(Er).

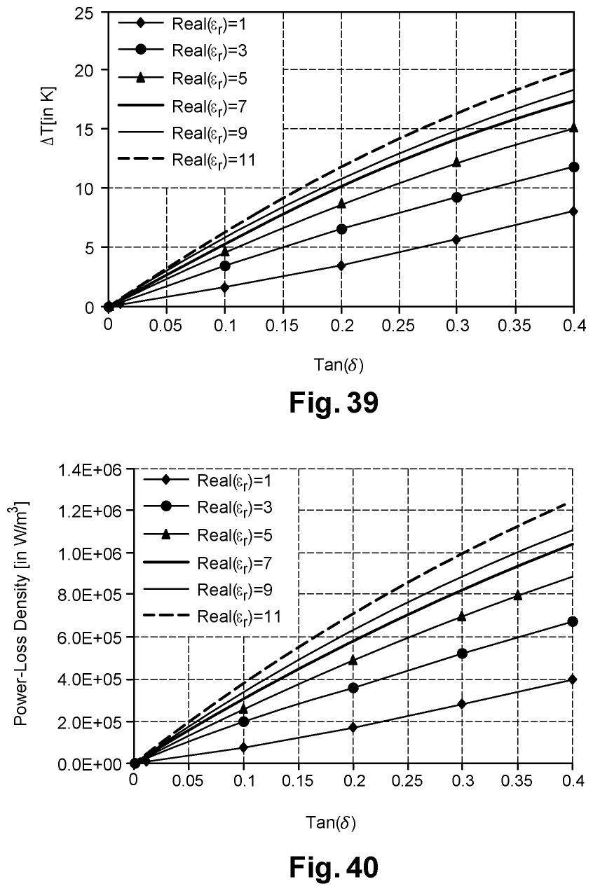

FIG. 39 illustrates the temperature change in the thermal indicator material as a function of the imaginary part Tan(delta).

FIG. 40 illustrates the power loss density in the thermal indicator material as a function of the imaginary part Tan(delta).

The imaging solution presented in relation with FIGS. 1 to 15 and 38 to 40 render possible the extraction of power-density and energy-density metrics as function of beamsteering angles from hybrid EM-Thermal sensing.

Porting of Spintronics hybrid Thermal-Electromagnetic sensing into advanced Silicon technologies (e.g., FD-SOI) platforms leads to co-integration of SCO materials with smart FEMs for replacing conventional IR-imagers by low-cost visible cameras with fluorescent functionalization processes.

Time-Domain based extraction of temperature distributions is possible at micronic and nano scale levels with accurate derivatives and integrals to accurately measure Entropy and Energy-based metrics.

Time-Domain broadband extractions of material properties can be obtained using extended Kronig-Kramers relations.

Advantageously, hybridization of antenna/probe solutions with SCO EM-Thermal conversions can be applied for measuring the radiation of circuits and systems.

Furthermore, the use of 3D conformal patterning combined with 3D conformal shielding strategies provides for improved EM-Thermal conversions.

While in the embodiments described above the testing is performed on a DUT, in other embodiments, the described imaging device could be used with Smart-Skins and clothes solutions for extracting human body and animals energy distributions using SCO materials.

Furthermore, the SCO EM-Thermal imaging solution described herein could be combined with Body-Biasing functionality for controlled sensitivity and dynamic ranges with improved signal to noise ratio.

### Second Aspect—Correlation-Based Entropy Extraction Solution for MIMO Systems

According to the first aspect described above, energy-density, power-density and/or entropy can be extracted based on detected temperature variations. Such metrics are useful for several reasons, not least because they permit an evaluation of physical parameters such as the SAR (Specific Absorption Rate).

Following the international standardization bodies, the specific absorption rate as a physical quantity to prevent excess temperature rise due to radio-frequency (RF) exposure can be extracted based on the following proportionality:

$$SAR = \frac{\partial T ( t )}{\partial t} {\vert}_{t = 0}$$

The physical properties of entropy in link with the second principle of thermodynamics creates a direct link between EM fields and their effects in living tissues. Correlating the temperature-based equation with the electromagnetic-based equation provides means for accurate SAR extraction both in frequency and time-domains:

$$SAR = \frac{1}{V} {\int }_{sample} \frac{σ ( r ) {\vert E ( r ) \vert}^{2}}{ρ ( r )} dr$$

where:
σ is the sample electrical conductivity;
E is the RMS (Root Mean Square) electric field;
ρ is the sample density; and
V is the volume of the sample.

Sensitivity analysis can be conducted based on the time evolution of the temperature biological bodies surface exposed to RF and Microwave electromagnetic fields fusing a primary delay function, as expressed by basic approximation equation:

$$T ( t ) = {T}_{max} ( 1 - {e}^{- \frac{t}{τ}} )$$

where Tₘₐₓ represents the maximum temperature elevation, τ being the thermal time constant. The initial temperature distribution can be related to the spatial gradient of the SAR distribution.

Furthermore, unified modeling and measurement extractions for convergence of Shannon's entropy and Boltzmann's entropy allow accurate extraction of key parameters characterizing the quality of RF wireless systems such as SNR, channel capacity, data rate and correlation between antennas in MIMO applications. Such unification will foster multi-physics characterization instruments.

Correlation techniques provide a useful tool for extracting parameters in wireless systems. Correlation techniques are for example described in more detail in the publication by Q. H. Tran, S. Wane, F. Terki, D. Bajon, A. Bousseksou, J. A. Russer, P. Russer, entitled “Toward Co-Design of Spin-Wave Sensors with RFIC Building Blocks for Emerging Technologies”, 2018 2nd URSI Atlantic Radio Science Meeting (AT-RASC), the contents of this publication being hereby incorporated by reference. Furthermore, it is possible to perform wireless measurements of power levels and energy density levels at DC and RF/Microwave frequencies, and entropy extraction, as described for example in more detail in the publication by S. Wane et al. entitled “Energy-Geometry-Entropy Bounds aware Analysis of Stochastic Field-Field Correlations for Emerging Wireless Communication Technologies”, URSI General Assembly Commission, New Concepts in Wireless Communications, Montreal 2017), the contents of this publication being hereby incorporated by reference.

Where equipment, such as a vector network analyzer (VNA), is available for S-parameter measurements, S-parameters-based extraction of antenna correlations can be obtained using the following equations:

$$ρ ( ω ) = \frac{\vert {S}_{11}^{*} ( ω ) {S}_{12} ( ω ) + {S}_{21}^{*} ( ω ) {S}_{22} ( ω ) \vert}{\sqrt{1 - {\vert {S}_{11} ( ω ) \vert}^{2} - {\vert {S}_{21} ( ω ) \vert}^{2}} \sqrt{1 - {\vert {S}_{22} ( ω ) \vert}^{2} - {\vert {S}_{12} ( ω ) \vert}^{2}}}$$

$${ρ}^{{η}_{1 \_} {η}_{2}} ( ω ) = \frac{ρ ( ω )}{\sqrt{( 1 - {η}_{1} )} \sqrt{( 1 - {η}_{2} )}}$$

where η₁ and η₂ are the radiation efficiencies of antennas 1 and 2 extracted from measurements for variable impedance matching, S₁₁, S₁₂, S₂₁, and S₂₂ are the S-parameters associated with the two-antenna network with antennas 1 and 2, and ω is the frequency.

However, S-parameters-based extraction of antenna correlations have limitations, and S-parameters are not always available. In particular, the measurement of S-parameters generally involves certain interactions with the DUT, which is not always possible.

An alternative solution based on stochastic field-field based correlation analysis is proposed hereafter, enabling the determination of energy metrics, based on the following formula (see also the publication by S. Wane, D. Bajon, J. Russer, P. Russer, and J. M. Moschetta, “Concept of Twin Antenna-Probe using Stochastic Field-Field X-Correlation for Energy Sensing and Low-Noise Blind Deconvolution”, IEEE Conference on Antenna Measurements & Applications Focus, Syracuse, 23-27 Oct. 2016., the contents of which are hereby incorporated by reference to the extent permitted by the law. Eᵢ (θ, φ) and Eⱼ (θ, φ) being the radiation patterns of antenna 1 and 2 respectively, the envelope cross-correlation between the two antenna 1 and 2 expressed in the frequency-domain is given by the following equation:

$$p ( ω ) = \frac{\vert {\int }_{4 π} d Ω {E}_{1} ( θ , ϕ ) \cdot {E}_{2}^{★} ( θ , ϕ ) \vert}{\sqrt{{\int }_{4 π} d Ω {\vert {E}_{1} ( θ , ϕ ) \vert}^{2}} \sqrt{{\int }_{4 π} d Ω {\vert {E}_{2} ( θ , ϕ ) \vert}^{2}}}$$

where Ω is the surface of a sphere.

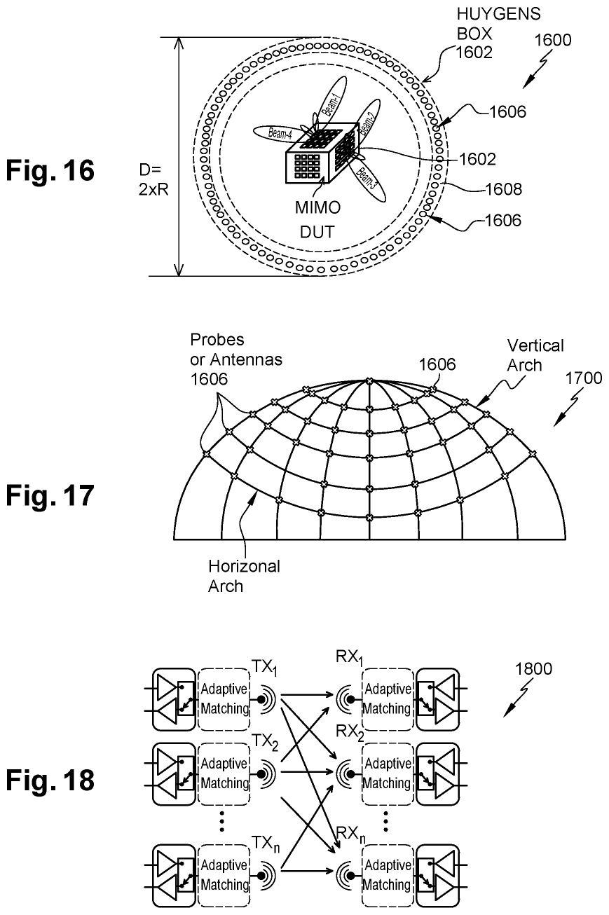

A test solution exploiting this correlation analysis will now be described in relation with FIGS. 16 to 20.

FIG. 16 illustrates a test environment 1600 for testing a MIMO (multiple-input multiple-output) DUT 1602 based on a spherical Huygens box 1604 of diameter D, and comprising antennas or probes 1606 suitable for detecting electric and/or magnetic fields. The Huygens box 1604 is hollow, and the DUT 1602 is positioned close to the center of the box 1604. The probes 1606 are for example sensor elements such as spin-wave elements, or any other element sensitive to RF and/or mmWave signals.

The MIMO DUT 1602 for example comprises multiple antennas emitting multiple beams, of which four are represented labelled Beam-1, Beam-2, Beam-3 and Beam-4.

The Huygens box 1604 for example comprises absorbers 1608 surrounding the probes.

FIG. 17 illustrates correlator spherical mapping 1700 according to an example embodiment of the present disclosure. For ease of illustration, only part of the sphere is represented. FIG. 17 illustrates in particular an example of the positioning of the probes or antennas 1606, which are for example at the intersection points of regularly spaced horizontal arches corresponding to lines of latitude, and vertical arches corresponding to meridian lines.

FIG. 18 schematically illustrates a MIMO link with scatterers in the presence of noise including transmitters TX₁ to TXₙ, receivers RX₁ to RXₙ, and TX and RX adaptive matching according to an example embodiment of the present disclosure.

It is proposed (see for example the publication by S. Wane, D. Bajon, J. Russer, P. Russer, and J. M. Moschetta, “Concept of Twin Antenna-Probe using Stochastic Field-Field X-Correlation for Energy Sensing and Low-Noise Blind Deconvolution”, IEEE Conference on Antenna Measurements & Applications Focus, Syracuse, Oct. 2016) that for any bounded system with Entropy SEntropy and rest Energy ERest there exists a universal upper limit on the entropy-to-energy ratio which leads to the following inequality accounting for geometry:

S_{Entropy}/E_{Rest}≤2πR_{Geometry}

where R=R_{Geometry} represents the radius of the sphere circumscribing the system.

For topologically compact systems, R is to be defined in terms of the system's volume.

In the derivation of (1) we have assumed h/2π=k=G=1 without loss of generality (units are scaled accordingly).

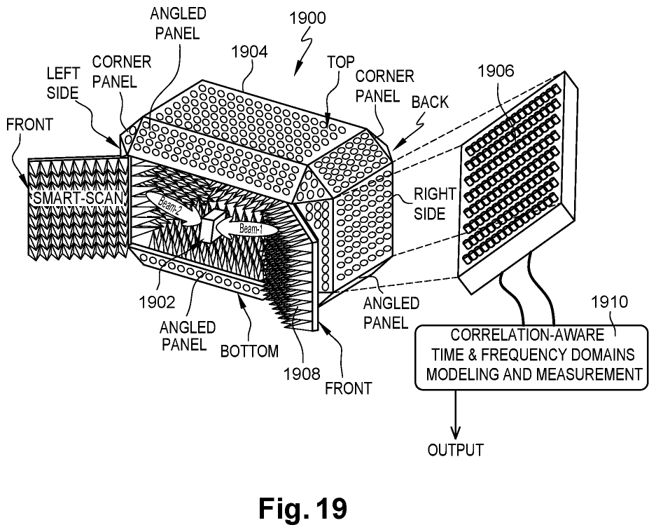

FIG. 19 illustrates a test system based on a Huygens box 1900 according to an example embodiment of the present disclosure. The Huygens box in the embodiment of FIG. 19 is substantially the shape of a rectangular parallelepiped, which is hollow, and comprises six main panels: a top panel (TOP); bottom panel (BOTTOM); left side panel (LEFT SIDE); right side panel (RIGHT SIDE); back panel (BACK), and front panel (FRONT). The main panels are all for example square or rectangular in shape. In the example of FIG. 19, the front panel is formed of hinged doors that open from the center to allow a DUT 1902 to be positioned inside the box.

In some embodiments, the box 1900 comprises further angled panels (ANGLED PANEL) at each intersection between a pair of the six main panels, such that there are no 90-degree corners on the box. There are for example twelve such angled panels, which are for example rectangular, and angled at substantially 45-degrees with respect to two main panels that they join with.

Furthermore, there is for example a corner panel (CORNER PANEL) present at each intersection of three angled panels, positioned at the corners of the rectangular parallelepiped.

Each of the main panels, angled panels, and corner panels comprises a probe array of two or more probes 1906. The probes 1906 are for example sensor elements such as spin-wave elements, or any other element sensitive to RF and/or mmWave signals. The angled panels and corner panels help to approximate a spherical surface for the probes 1906. The probes 1906 are for example positioned on each panel such that they receive electromagnetic signals emitted by antennas of the DUT 1902. The box 1900 is for example lined with absorbers 1908.

Each of the probes 1906 of each of the panels is for example coupled to a device 1910 capable of determining parameters of the DUT 1902 based on signals captured by the probes. In some embodiments, the device 1910 is a correlation-aware time and frequency domains modeling and measurement device (CORRELATION-AWARE TIME & FREQUENCY DOMAINS MODELING AND MEASUREMENT). The device 1910 for example comprises a processing device, such as an ASIC, FPGA or one or more processing units under control of instructions stored in an instruction memory. The device 1910 is for example configured to process signals sampled simultaneously by selected pairs of probes (i.e. twin antenna probe elements) in order to extract, based on correlation techniques, energy-density, power-density and/or entropy values in relation with the signals emitted by the DUT 1902. A switching matrix capable of simultaneously capturing signals from a pair of probes in a probe array is described for example below, and also in the PCT application entitled “Full-Crossover Multi-channel switching matrix for MIMO circuits and systems operating in time and frequency domains” and published WO2021/123447, the contents of which is hereby incorporated by reference.

The proposed concept of X-Correlation processing relies on simultaneously probing the EM fields with the twin antenna probe elements. The correlation calculations of the disclosure allow efficient noise reductions as explained in the following equations. A non-normalized cross-correlation function may be expressed by a cross-correlation C_{AB} (τ) of stationary stochastic signals S_{A} (t) and S_{B} (t) such as the intensities of the different pixels. The cross-correlation is defined by the following equation where the brackets denote the ensemble average:

$${C}_{AB} ( τ ) = 〈 {S}_{A} ( t ) \vert {S}_{B} ( t + τ ) 〉 = {lim}_{T \to } \frac{1}{2 T} {\int }_{- T / 2}^{+ T / 2} {S}_{A} ( t ) {S}_{B} ( t + τ ) dt$$

where T is a period of measurement.

The proposed concept of X-Correlation processing relies on simultaneously probing the EM Fields with the Twin Antenna Probe elements. When the signals and the noise contributions are uncorrelated then applying the Esperance operator E[.], the following equation can be derived:

$$E [ ( {X}_{A} + {N}_{A} ) \overline{( {X}_{A} + {N}_{B} )} ] = E [ ( {X}_{B} + {N}_{A} ) \overline{( {X}_{B} + {N}_{B} )} ] = E [ {\vert {X}_{A} \vert}^{2} ] + E [ {X}_{A} \overline{{N}_{B}} ] + E [ {N}_{A} \overline{{X}_{A}} ] + E [ {N}_{A} \overline{{N}_{B}} ] = E [ {\vert {X}_{B} \vert}^{2} ] + E [ {X}_{B} \overline{{N}_{A}} ] + E [ {N}_{B} \overline{{X}_{B}} ] + E [ {N}_{B} \overline{{N}_{A}} ] = E [ {\vert {X}_{A} \vert}^{2} ] = E [ {\vert {X}_{B} \vert}^{2} ]$$

where N_{A} and N_{B} are the noise contributions on the different probes.

The correlation matrix can be expressed as function of the time-windowed signal S_{T} (t):

$$C ( ω ) = ( \begin{matrix}{C}_{11} ( ω ) & {C}_{12} ( ω ) & ⋯ & {C}_{1 N} ( ω ) \\ {C}_{21} ( ω ) & {C}_{22} ( ω ) & ⋯ & {C}_{2 N} ( ω ) \\ ⋮ & ⋮ & ⋯ & ⋮ \\ {C}_{N 1} ( ω ) & {C}_{N 2} ( ω ) & ⋯ & {C}_{N N} ( ω )\end{matrix} )$$

$$C ( ω ) = {lim}_{T \to ∞} \frac{1}{2 T} 〈 {S}_{T} ( t ) \vert {S}_{T}^{†} ( t + τ ) 〉$$

The superscript † refers to Hermitian conjugate operation.

Wavelet multiresolution analysis is proposed for simultaneous identification and localization of noisy sources for EMC/EMI applications based on Energy density and Entropy considerations. Field-Field correlation analysis represents a powerful tool based on physical considerations for relating energy, entropy and geometry. In its exhaustive form, the holographic principle is a bridge between the geometry and information content of space-time.

For deterministic noise power density distribution, the challenge of energy detection of unknown signals in presence of noise is discussed in the publication S. Wane, D. Bajon, J. Russer, P. Russer, and J. M. Moschetta, “Concept of Twin Antenna-Probe using Stochastic Field-Field X-Correlation for Energy Sensing and Low-Noise Blind Deconvolution”, IEEE Conference on Antenna Measurements & Applications Focus, Syracuse, Oct. 2016.

For stochastic signals, it is established that numerical values of noise amplitudes cannot be specified. Thus, for modeling and measuring stochastic signals, it is proposed to deal with energy and power spectra. The power spectra of the signals can be deduced from the correlation matrix C(ω).

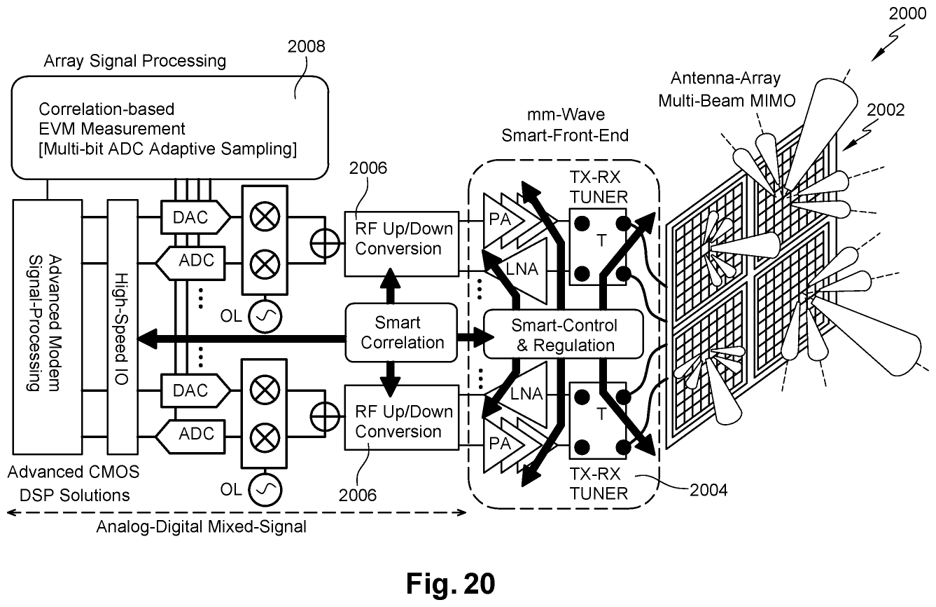

FIG. 20 schematically illustrates a detection system 2000. The system 2000 for example comprises an antenna array 2002, which is for example a multi-beam MIMO. A plurality of selected antennas are for example coupled to a Front-end module 2004, which for example comprises a pair switchs T for coupling the antennas to power amplifiers PA for transmission, or to low noise amplifiers LNA for reception. RF Up/Down converters 2006 are coupled between the front-end module and a mixing stage, and two-way analog/digital conversion stages, each comprising an ADC and DAC, are in turn coupled to the mixing stage. A high-speed input/output interface is coupled to the analog/digital conversion stages and an advanced modem signal processing circuit is for example coupled to the high-speed input/output interface. A correlation-based EVM measurement circuit 2008, based on the techniques described herein, is for example coupled to the ADCs, DACs, and to advanced modem signal processing stage, and for example permits array signal processing in order to extract parameters as described herein.

The beamformer system is composed, in one example, of 8×8=64 antennas functioning in the band 26 GHz-30 GHz for mobile telephones, base-stations and SATCOM. This solution provides for example very fast and easy detection of faulty antenna elements/beam-former chips: [Interferometric EM-Thermal Measurement for Vectorial Characterizations]. For example, one pratical application is for industrial testing of beamforming circuits and modules.

This invention supports 3D Near-Field and Far-Field Scanning system for DC, RF, mmWave/Optical applications based on the following functionalities:
Synchronized Vectorial Probes [including X, Y and Z polarizations] positioned in linear-array segments for Near-Field and Far-Field Sensing and Imaging; and
Angular Rotary-System with controlled speed for High-Resolution accurate extraction of Near-Field and Field amplitude and phase information with associated smart Signal-Processing.

Among the possibilities enabled by this invention include: 3D Near-Field and Field Sensing & Imaging:
EM/EMC EMI Fields Maps in Time & Frequency domains
Radiation patterns
Holographic Thermal-Visual Imaging

### Third Aspect—Correlation-Based Time-Reversal Calibration Solution

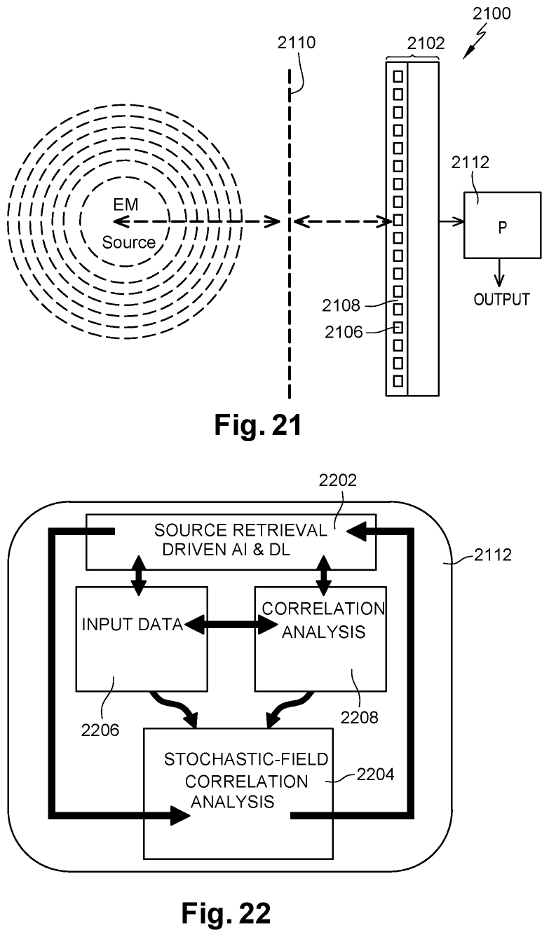

The techniques described in relation with FIGS. 16 to 20 permit parameters of a RF or mmWave transmission system to be extracted, including energy-density, power-density, and/or entropy at a given distance and location from the EM source. However, for some applications, it would be desirable to be able to determine the field present at the source, or at an intermediate point between the detection device and the EM source, without bringing probes closer to the EM source. Indeed, such information can permit the field at other distances to be deduced. A solution proposed herein is to use a time-reversal technique, as will now be described in more detail with reference to FIGS. 21 to 27.

FIG. 21 illustrates a test environment 2100 for correlation-based time-reversal calibration solution for probe-array systems using artificial intelligence according to an example embodiment of the present disclosure. A detection device 2102 is distanced from an EM source generating a Stochastic field represented by dashed circles in FIG. 21. The detection device 2102 comprises a detection array of probes 2106 surrounded for example by absorbers 2108. It is for example desired to perform EP-field plane sampling in a plane 2110 between the EM source and the probe array of the detection device 2102. The detection device 2102 is coupled to a processing device 2112 configured to receive samples from the detection device 2102, and to generate an output (OUTPUT) indicating the electric field present at intermediate points, such as in the plane 2110. The processing device 2112 for example comprises an ASIC, FPGA, and/or one or more processing units under control of instructions stored in an instruction memory.

FIG. 22 schematically illustrates modules of the processing device 2112 for correlation-based time-reversal calibration solution for probe-array systems using artificial intelligence according to an example embodiment of the present disclosure.

The processing device 2112 comprises a source retrieval module (SOURCE RETRIEVAL DRIVEN AI & DL) 2202, which is for example driven by Artificial Intelligence (AI) and Deep-Learning (DL), and a stochastic-field correlation analysis module (STOCHASTIC-FIELD CORRELATION ANALYSIS) 2204, that uses Time-Reversal and Back-Propagation Algorithms. An input data module (INPUT DATA) 2206 is for example configured to provide input data based on modeling or measurement of EM fields. A correlation analysis module (CORRELATION ANALYSIS) 2208 is for example configured to perform correlation analysis based on modeling or measurement of EM fields.

The Time-Reversal and Back-Propagation algorithms are based on the following principles. The derivative of the cross-correlation functions between two sampling points A (in channel 1) and B (in channel 2) in noisy environment as function of Cardinal Sine function (referenced as Sinc) law is extracted based on the following expression:

$$\frac{d}{dt} C ( τ , A , B ) = {K}_{Noise} [ G ( τ , A , B ) - G ( - τ , A , B ) ] ∝ Sinc ( \frac{ω r}{c} )$$

where:
c is the speed of light and r represents the separation distance between the points A and B at frequency ω.
K_{Noise} is relative to the ambient noise. This equation refers to free space. For a medium different from free space the argument of the Sinc function will incorporate a propagation constant function of the medium properties.
G represents the Green's function retrieved by cross-correlating fluctuations recorded at two locations A and B. This energy balance provides time-reversal conditions for proper retrieval of time-domain Green's function between two points by performing a cross-correlation of the ambient noise field received on two sampling points τ is a positive time shift and —τ is a negative time shift.

We use Cross-Entropy metrics for evaluating the accuracy of the stochastic measurements:

$$Cross - Entropy = - {\sum }_{u = 0}^{N} {\sum }_{v = 0}^{M} Iu , vlog ( Pu , v )$$

where:
Iu,v denotes the true value i.e. 1 if sample u belongs to class v and 0 otherwise.
Pu,v the probability predicted by for sample u belonging to class v.

For two random variables I_{S}_{₁} and I_{S}_{₂} with finite variances, the correlation of them is defined as:

$${CC}_{{I}_{{S}_{1}} {I}_{{S}_{2}}} = \frac{Cov ( {I}_{{S}_{1}} {I}_{{S}_{2}} )}{σ ( {I}_{{S}_{1}} ) σ ( {I}_{{S}_{2}} )}$$

with the covariance:

Cov(I_{S}_{₁} I_{S}_{₂})=E[(I_{S}_{₁}−μ1)(I₂−μ2)]

where μi and σ(I_{S}_{ᵢ}) are the expectation and standard deviation of I_{S}_{ᵢ} i=1, é. Here

$$\\ {CC}_{{I}_{{s}_{1}} {I}_{{s}_{2}}}$$

denotes a coefficient number in the interval [−1, +1]. The boundaries −1 and +1 will be reached if and only if I_{S}_{₁} and I_{S}_{₂} are indeed linearly related. The greater the absolute value of

$$\\ {CC}_{{I}_{{s}_{1}} {I}_{{s}_{2}}}$$

the stronger the dependence between X1 and X2 is.

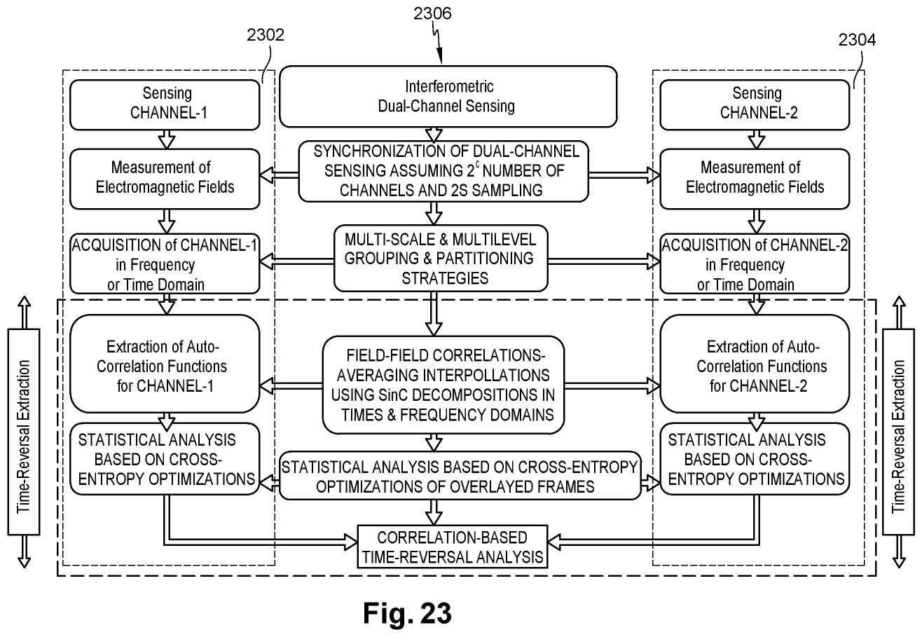

FIG. 23 illustrates operations in a method of correlation-based time-reversal according to an example embodiment of the present disclosure. Two similar methods 2302, 2304 are for example executed in parallel in order to process channel 1 and channel 2 signals. Furthermore, some common processing operations 2306 are for example performed in parallel.

The method 2302 for example comprises the following steps:
sensing channel-1;
measurement of electromagnetic fields;
acquisition of channel-1 in frequency or time domain; and
time-reversal extraction, comprising:
   extraction of auto-correlation functions for channel-1; and
   statistical analysis based on cross-entropy optimizations.

The method 2304 comprises similar steps for channel-2. The methods 2302 and 2304 are followed by a common step of correlation-based time-reversal analysis.

The common processing operations 2306 for example comprise:
interferometric dual-channel sensing;
synchronization of dual-channel sensing assuming a 2^{c} number of channels and 2S sampling, for supporting the measurement operations of channels 1 and 2;
multi-scale and multi-level grouping and partitioning strategies, for supporting the acquisitions of the channels 1 and 2 in frequency or time domain; and
as part of the time-reversal extraction:
   field-field correlations-averaging interpollations using SinC decompositions in time and frequency domains; and
   statistical analysis based on cross-entropy optimizations of overlayed frames.

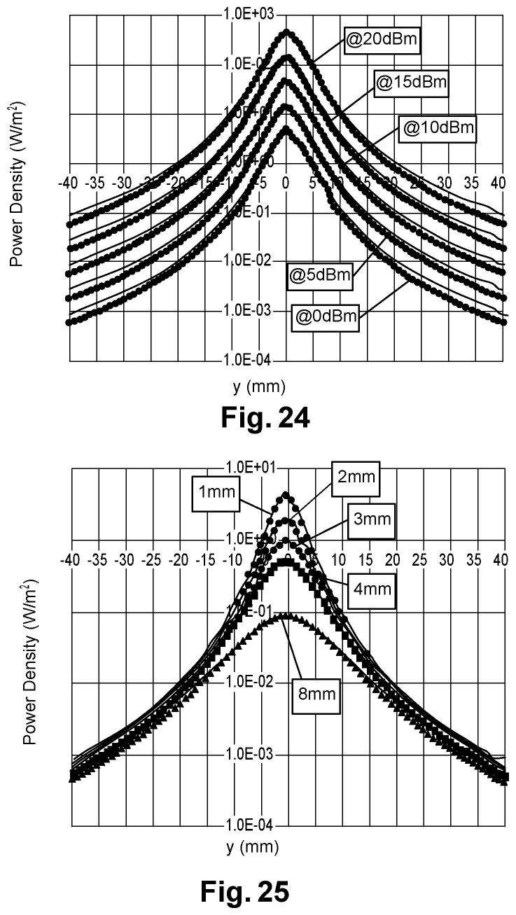

FIG. 24 is a graph representing power-density as a function of radiated power in the test environment of FIG. 21 according to an example embodiment of the present disclosure. FIG. 24 illustrates in particular theoretical values by dotted curves, and the corresponding values generated by the time-reversal techniques described herein, for five transmission powers at 0 dBm, 5 dBm, 10 dBm, 15 dBm and 20 dBm.

FIG. 25 is a graph representing power-density as a function of a distance to a probe array in the test environment of FIG. 21 according to an example embodiment of the present disclosure. FIG. 25 illustrates in particular theoretical values by dotted curves, and the corresponding values generated by the time-reversal techniques described herein, for five distances of 1 mm, 2 mm, 3 mm, 4 mm and 8 mm from the EM source.

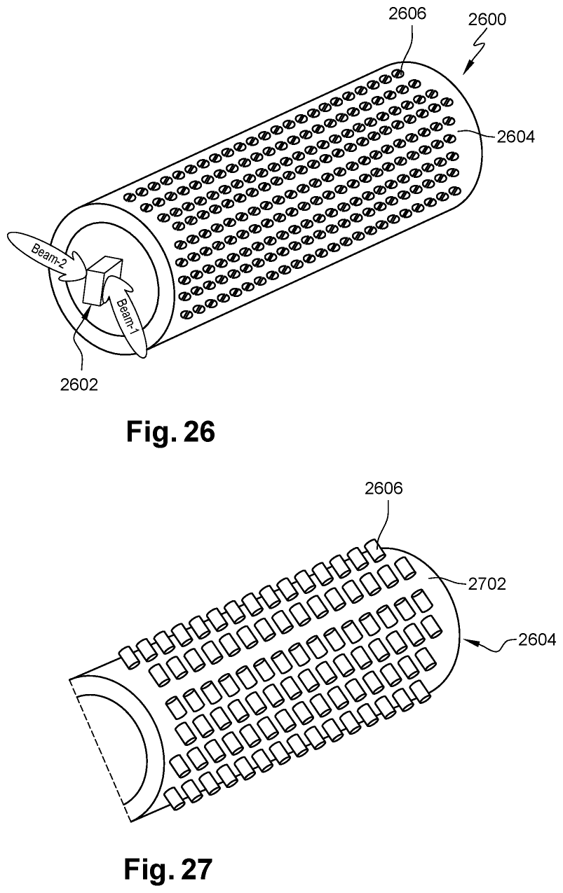

FIG. 26 illustrates a MIMO test solution 2600 using smart anechoic chambers with a tunable probe array according to an example embodiment of the present disclosure. FIG. 26 illustrates in particular a DUT 2602 and an anechoic chamber 2604, which is substantially cylindrical in shape, and is hollow such that a DUT 2602, which is for example a MIMO device, can be placed inside the chamber 2600. Probes 2606 are located around the cylindrical wall of the chamber 2604, so as to detect signals emitted by the DUT 2602.

FIG. 27 illustrates the MIMO test solution 2600 of FIG. 26 in more detail. FIG. 27 illustrates in particular an interior surface 2702 of the anechoic chamber 2604, and shows the probes 2606 in more detail. The orientations of the probes 2606 are not represented precisely in FIG. 27, the probes for example being orientated such that they detect signals originating from around the central axis of the cylinder.

### Fourth Aspect—Cognitive Correlators Including Multi-Scale and Multi-Level Group-Sampling in Time and Frequency-Domains

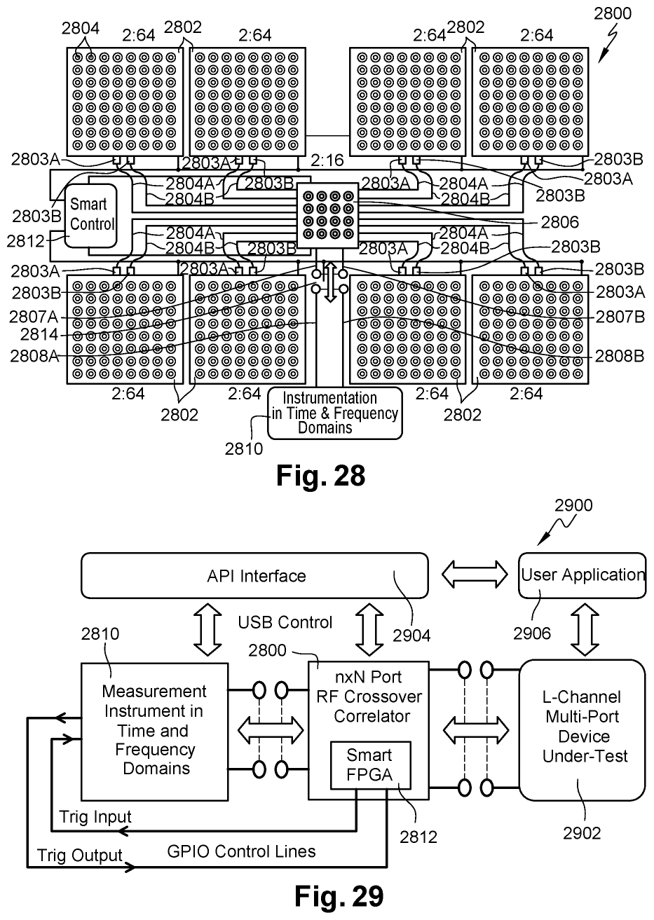

The correlation techniques described herein are based on dual channel simultaneous readings of probe pairs in multiple arrays. A device capable of performing such sampling in time and frequency domains will now be described with reference to FIGS. 28 to 36.

FIG. 28 schematically illustrates a switching system 2800, based on a lego-mosaic approach, for MIMI systems according to an example embodiment of the present disclosure. The switching system 2800 for example comprises matrices 2802 that are assembled to form an array of a desired size. In the example of FIG. 28, there are eight matrices 2802, although it will be apparent from the description hereafter that many other numbers of matrices would be possible.

Each matrix 2802 is for example a device as described in more detail in the PCT application entitled “Full-Crossover Multi-channel switching matrix for MIMO circuits and systems operating in time and frequency domains” and published as WO2021/123447. Each matrix 2802 for example comprises a plurality N of input/output ports 2804, each of which is for example coupled to a corresponding antenna or probe (not illustrated in FIG. 28). In some embodiments, each input/output port 2804 provides a signal on a single conductor or wire, while in other embodiments there may be multiple conductors or wires provided by each input/output port 2804. In the example of FIG. 28, each matrix 2804 comprises 64 such input/output ports arranged in an 8 by 8 sub-array, although in alternative embodiments each matrix 2804 could comprise a different number N of input/output ports 2804 and/or a different arrangement of the ports.

Each matrix 2802 further comprises a plurality M of input/output ports 2804. In the example of FIG. 28, each matrix 2802 comprises two input/output ports 2804, respectively labelled 2803A and 2803B, although in alternative embodiments there could be a different number, depending on the number of channels to be detected simultaneously. The input/output ports 2804 are each coupled, via corresponding lines, to a matrix interconnect 2806. The matrix interconnect 2806 for example provides an interface between the M input/output lines of each of the matrices 2802, which correspond to a total of J input/output ports, and K global input/output ports 2807, there being two such ports 2807A, 2807B in the example of FIG. 28. The J input/output ports of the matrix interconnect 2806 are for example coupled to instrumentation 2810 for sampling, in time and frequency, signals received via the switching system 2800, and in particular via the input/output ports 2804. The number J of input/output ports is for example equal to the number M of input/output ports, and is for example equal to the number of channels. In the example of FIG. 28, the two input/output ports 2807A, 2807B of the switching system 2800 are coupled to the instrumentation 2810 via corresponding lines 2808A and 2808B, and for example via an amplitude adaptation circuit 2814.

The system 2800 further comprises, for example, a control circuit (Smart Control) 2812 for controlling each of the matrices 2802, and the matrix interconnect 2806. In some embodiments, the control circuit 2812 is configured to synchronize the electrical coupling of the one or more selected ones of the N input/output ports of each panel to one or more of the K input/output ports. In the example of FIG. 28, the control circuit 2812 is configured to synchronize the electrical coupling of two of the N input/output ports, each of which may be present in any of the matrices 2802, to the two ports 2807A, 2807B respectively. This coupling for example permits a simultaneous sampling of the signals present at the selected input/output ports 2804.

According to some embodiments, N, M, J and/or K are integers, each equal to a power of 2.

According to some embodiments, N is equal to at least 16, M is equal to 2 or 4, J is equal to at least 4, and K is equal to 2 or 4.

According to some embodiments, M and K are equal.

According to some embodiments, the synchronization is performed for amplitude and phase, such that the amplitudes of the propagated signals are substantially equal, and a time delay of the transmission path between each input/output port 2804 of each channel is substantially equal.

According to some embodiment, the control circuit 2812 is a programmable circuit, such as an FPGA.

According to one embodiment, the control circuit 2812 is configured to communicate with matrix control circuits of each matrix in order to perform the synchronization.

According to one embodiment, the N input/output ports of each panel is configured to receive a signal at a frequency of up to 30 GHz, and in some embodiments of up to 64 GHz or more.

According to one embodiment, the switch matrix system further comprising an amplitude adaptation circuit 2814 configured to adapt an amplitude of signal present at the K input/output ports, for example based on a control signal received from a driver circuit of a measurement apparatus coupled to the K input/output ports, the amplitude adaptation circuit for example comprising one or more amplifiers and/or attenuators, and for example at least one amplifier and/or attenuator for each channel.

According to one embodiment, the K input/output ports are configured to be coupled to input/output ports of an oscilloscope.

According to a further aspect, a method is for example performed using the above switching system, involving coupling a plurality of sensors to a measurement apparatus/instrumentation using the switching system.

FIG. 29 schematically illustrates a test system 2900 based on the switching system 2800 of FIG. 28 according to an example embodiment of the present disclosure. The test system 2900 for example comprises an L-channel multi-port DUT 2902, having its output ports coupled to the nxN input/output ports 2804 of the switching system 2800, where n is equal to the number of matrices 2802. The control circuit 2812 is implemented for example by an FPGA, such as a smart FPGA. The K input/output ports 2807 of the switching system 2800 are coupled to the instrumentation 2810, which for example comprises a VNA and/or oscilloscope. The control circuit 2812 is also for example coupled to the instrumentation 2810 via GPIO Control lines including a Trig Input line and a Trig Output line, these signals being described in more detail in the PCT publication WO2021/123447.

In some embodiments, the instrumentation 2810, and the switching system 2800, are coupled to an API Interface 2904, which is, for example in turn coupled to a User Application 2906.

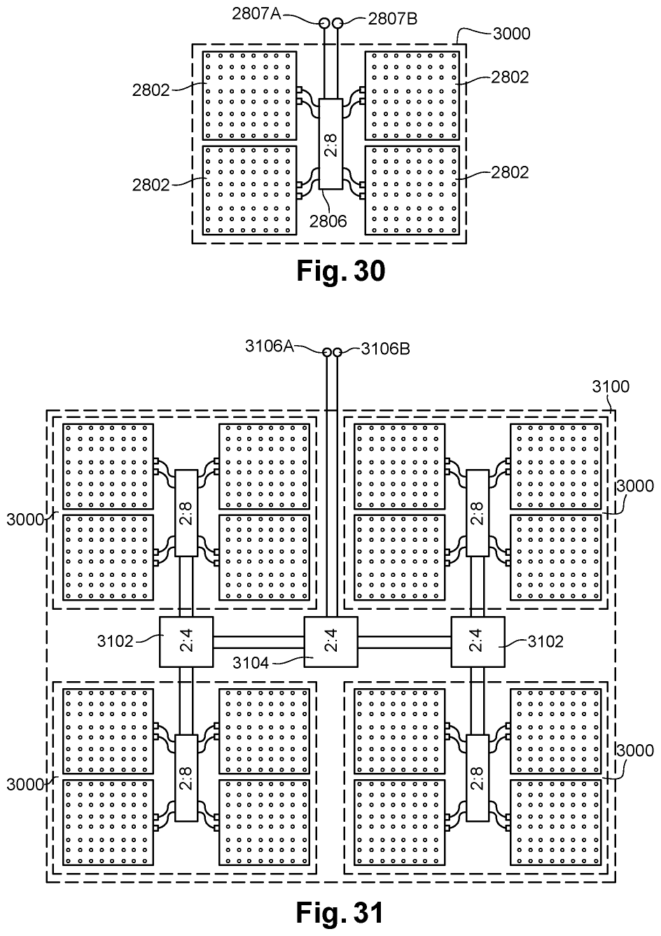

The modular scalability of the solution will be apparent from FIGS. 30 to 32.

FIG. 30 schematically illustrates a four-matrix MIMO array switching system 3000 of matrices 2802 interconnected by a matrix interconnect in similar manner to what is described in relation with FIG. 28. The output ports 2807A, 2807B can be coupled to the instrumentation 2810, or a further interconnect as illustrated in FIG. 31.

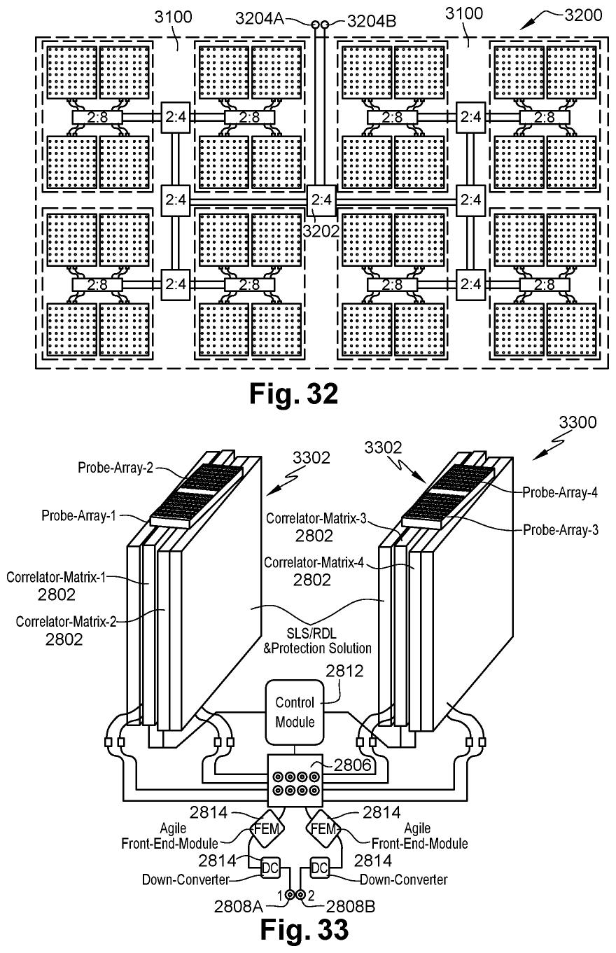

FIG. 31 schematically illustrates a 16-matrix, 32×32 MIMO array switching system 3100 comprising two of the systems 3000 of FIG. 30 coupled to a matrix interconnect 3102, and a further two of the systems 3000 of FIG. 30 coupled to a further matrix interconnect 3102, the matrix interconnects 3102 each being coupled to a matrix interconnect 3104. Output ports 3106A, 3106B of the matrix interconnect 3104 can be coupled to the instrumentation 2810, or a further interconnect as illustrated in FIG. 32.

FIG. 32 illustrates schematically a 32-matrix 64×32 MIMO array switching system 3200 comprising two of the systems 3100 of FIG. 31 coupled to a matrix interconnect 3202. Output ports 3204A, 3204B of the matrix interconnect 3202 can be coupled to the instrumentation 2810, or a further interconnect (not illustrated).

The modular interconnections of matrices as illustrated in FIGS. 30 to 32 can be extended as much as desired, based on the number of probes of the probe array or arrays.

FIG. 33 illustrates a MIMO array switching system 3300 comprising two 2-matrix modules 3302 according to an example embodiment of the present disclosure. Each module 3302 comprises, in addition to the 2-matrix modules, which are for example stacked back-to-back, i.e. with their input/output ports 2804 facing outwards, a corresponding probe array for each matrix, and an SLS/RDL (Selective Laser Sintering/Redistribution layer) device for coupling each probe array to the corresponding matrix. The SLS/RDL devices for example sandwich the matrices 2802 in each module 3302.

In the example of FIG. 33, the amplitude adaptation circuit 2814 comprises, for each output line 2808A, 2808B, a frond-end module FEM configured to perform the amplitude adaptation, and a down-converter configured to down convert the frequency of the received signal.

FIG. 34 illustrates a MIMO array switching system 3400 comprising a 16-matrix module according to an example embodiment of the present disclosure, on which is mounted an array of 128 smart-antennas and probes. The stack for example has a relatively low width w thanks to optimized metal-work and RDL connectivity.

FIG. 35 is a cross-section view of a re-distribution layer 3500 for MIMO systems according to an example embodiment of the present disclosure.

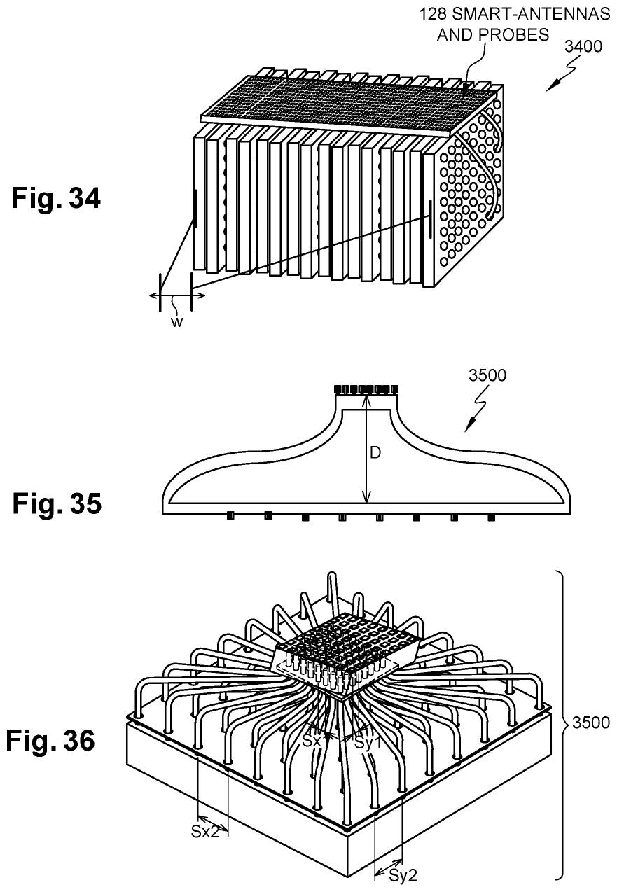

FIG. 36 is a perspective view of the re-distribution layer 3500 of FIG. 35 according to an example embodiment of the present disclosure.

The re-distribution layer 3500 is for example provided in the modules 3302 or 3400 of FIG. 33 or 34, for providing a connection interface between the probes of the probe array and the matrices 2802. In particular, the RDL 3500 for example provides a means for interfacing a pitches Sx1 and Sy1 of the probes of a probe array in the x and y directions, with pitches Sx2, Sy2 respectively of the input/output ports of the matrices. Such an RDL is for example described in more detail in the PCT application no. PCT/EP2021/064456, filed on 28 May 2021, the contents of which is hereby incorporated by reference.

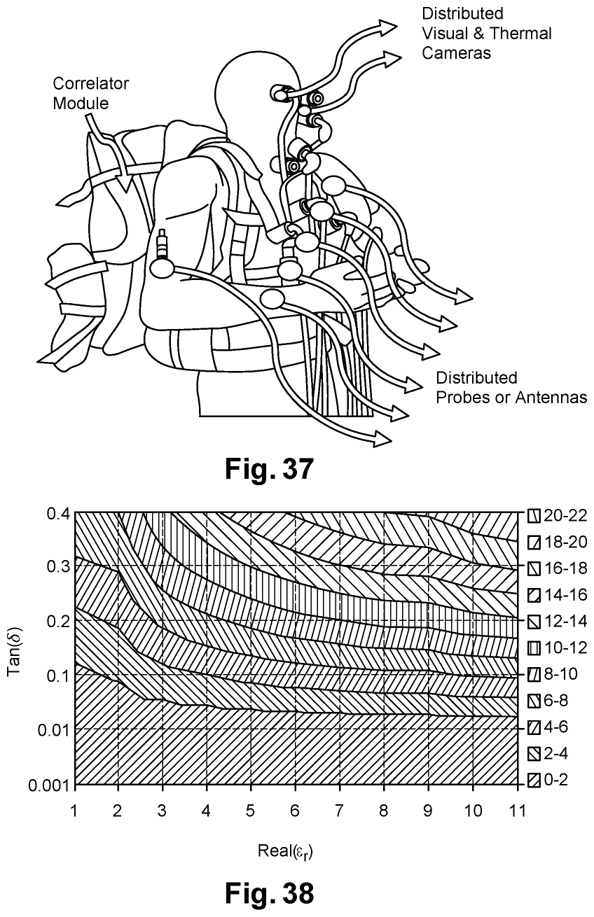

FIG. 37 illustrates an equipped robot or person according to an example embodiment. For example, headwear comprises visual and thermal cameras, probes/antennas are distributed in clothing, as part of a backpack and/or in an arm band, and the probes are coupled to a correlator module comprising, for example, the solutions for MIMO sensing and correlation techniques described herein. For example, these probes permit the measurement of an amount of exposure to multi-physics waves, including electromagnetic waves, thermal wave and/or sound waves. For example, such a solution could have application for workers in hazardous environments, including industrial environments, but also for persons while at home, travelling by car, plane or boar, etc.

Various embodiments and variants have been described. Those skilled in the art will understand that certain features of these embodiments can be combined and other variants will readily occur to those skilled in the art.

Finally, the practical implementation of the embodiments and variants described herein is within the capabilities of those skilled in the art based on the functional description provided hereinabove.

1. An electromagnetic-thermal sensing system comprising:
a conversion device configured to receive one or more electromagnetic signals emitted by a DUT the conversion device comprising a thermal indicator layer of quantum spin cross-over (SCO) material configured to change temperature as a function of an electrical and/or magnetic field present at the thermal indicator layer; and
an imaging device configured to capture one or more images of the conversion device.

2. The electromagnetic-thermal sensing system of claim 1, further comprising a processing device configured to determine, based on the one or more images, one or more temperature variations in the thermal indicator layer, and to determine one or more energy density values, power density values or entropy values based on the one or more temperature variations.

3. The electromagnetic-thermal sensing system of claim 1, wherein the imaging device is an infrared (IR) imaging device.

4. The electromagnetic-thermal sensing system of claim 1, wherein the imaging device is a visible light imaging device, and the conversion device further comprises a functional coating on a side facing the imaging device, the functional coating being configured to change color as a function of temperature.

5. The electromagnetic-thermal sensing system of claim 4, wherein the conversion device is integrated with the imaging device.

6. The electromagnetic-thermal sensing system of claim 4, comprising a further imaging device, configured to capture one or more images of the conversion device, wherein the further imaging device is an IR imaging device.

7. The electromagnetic-thermal sensing system of claim 1, wherein the conversion device further comprises one or more probe or antenna sensors for calibration purposes.

8. The electromagnetic-thermal sensing system of claim 1, wherein the conversion device is patterned with through holes.

9. A test system comprising the electromagnetic-thermal sensing system of claim 1 and the DUT, the electromagnetic-thermal sensing system being configured to sensing electromagnetic emissions from one or more antennas of the DUT.

10. The test system of claim 9, wherein a distance between the DUT and the electromagnetic-thermal sensing system is between 3 and 20 mm.

11. A method of electromagnetic-thermal sensing comprising:
receiving, by a conversion device, one or more electromagnetic signals emitted by a DUT, the conversion device comprising a thermal indicator layer of quantum spin cross-over (SCO) material configured to change temperature as a function of an electrical and/or magnetic field present at the thermal indicator layer; and
capturing one or more images of the conversion device using an imaging device.

12. The method of claim 11, further comprising:
determining, by a processing device based on the one or more images, one or more temperature variations in the thermal indicator layer; and
determining, by the processing device, one or more energy density values, power density values or entropy values based on the one or more temperature variations.
